(19) United States
(12) Patent Application Publication
(10) Pub. No.: US 2018/0000953 A1
(43) Pub. Date: Jan. 4, 2018

(54) LIPID NANOPARTICLE COMPOSITIONS

(71) Applicant: Moderna Therapeutics, Inc., Cambridge, MA (US)

(72) Inventors: Orn ALMARSSON, Shrewsbury, MA (US); Ciaran Patrick LAWLOR, Cambridge, MA (US)

(21) Appl. No.: 15/545,392

(22) PCT Filed: Jan. 21, 2016
(86) PCT No.: PCT/US2016/014280

§ 371 Date: Jul. 21, 2017

Related U.S. Application Data
(60) Provisional application No. 62/106,124, filed on Jan. 21, 2015.

(51) Int. Cl. A61K 47/54; A61K 31/7105; A61K 31/685; A61K 9/51; B82Y 5/00; A61K 48/00

(52) U.S. Cl. CPC A61K 47/544; B82Y 5/00; A61K 48/005; A61K 48/0008; A61K 47/542; A61K 31/685; A61K 9/5123; A61K 31/7105

(57) ABSTRACT

Disclosed herein are nanoparticle compositions including an mRNA and a lipid component and methods of using the same. The present invention provides compositions and methods involving lipid-containing nanoparticle compositions to deliver mRNA to cells. In one aspect, the invention provides a nanoparticle composition including (i) a lipid component including a phospholipid (which may or may not be unsaturated), a PEG lipid, a structural lipid, and a compound of formula (I) and (ii) an mRNA encoding a polypeptide of interest.

### TECHNICAL FIELD

The present disclosure provides compositions and methods using lipid nanoparticle compositions to deliver mRNA to and/or produce polypeptides in mammalian cells. In addition to mRNA, the lipid nanoparticle compositions of the invention may include cationic and/or ionizable amino lipids, phospholipids including polyunsaturated lipids, PEG lipids, and structural lipids in specific fractions.

### BACKGROUND OF THE INVENTION

In recent years, nucleic acids have increasingly been looked to as possible therapeutic agents. Therapeutic uses of messenger ribonucleic acid (mRNA) are particularly sought as an mRNA could be designed to encode a wide variety of polypeptides for many applications. For example, many diseases, disorders, and conditions, including cystic fibrosis, are characterized by aberrant protein activity and/or protein deficiency. It is theorized that the introduction of an appropriate mRNA could be translated within a cell to generate a polypeptide to replace, subvert, or otherwise combat an aberrant species. mRNA delivery systems could also be used to regulate important polypeptides such as vascular endothelial growth factor (VEGF), the transient and targeted expression of which is posited to combat stenosis in renovascular structures. Disruption of translational machineries by the introduction of non-translatable mRNA may also be feasible. However, the delivery of therapeutic RNAs to cells is made difficult by the relative instability and low cell permeability of RNAs. Thus, there exists a need to develop methods and compositions to facilitate the delivery of RNAs such as mRNA to cells.

Lipid-containing nanoparticle compositions have proven effective as transport vehicles into cells and/or intracellular compartments for a variety of RNAs. These compositions generally include one or more “cationic” and/or ionizable lipids, phospholipids including polyunsaturated lipids, structural lipids (e.g., sterols), and lipids containing polyethylene glycol (PEG lipids). Cationic and/or ionizable lipids include, for example, amine-containing lipids that can be readily protonated. Though a variety of such lipid-containing nanoparticle compositions have been demonstrated, improvements in safety, efficacy, and specificity are still lacking.

### SUMMARY OF THE INVENTION

The present invention provides compositions and methods involving lipid-containing nanoparticle compositions to deliver mRNA to cells.

In one aspect, the invention provides a nanoparticle composition including (i) a lipid component including a phospholipid (which may or may not be unsaturated), a PEG lipid, a structural lipid, and a compound of formula (I)

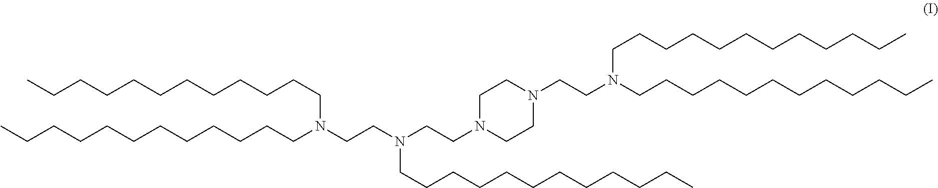 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and (ii) an mRNA encoding a polypeptide of interest.

In another aspect, the invention provides a method of producing a polypeptide of interest in a cell (e.g., a mammalian cell) involving contacting the cell with a nanoparticle composition including (i) a lipid component including a phospholipid (such as a polyunsaturated lipid), a PEG lipid, a structural lipid, and a compound of formula (I) and (ii) an mRNA encoding the polypeptide of interest, whereby the mRNA is capable of being translated in the cell to produce the polypeptide.

In yet another aspect, the invention provides a method of delivering an mRNA to a cell (e.g., a mammalian cell) involving administering to a subject (e.g., a mammal) a nanoparticle composition including (i) a lipid component including a phospholipid (such as a polyunsaturated lipid), a PEG lipid, a structural lipid, and a compound of formula (I) and (ii) an mRNA, in which administering involves contacting the cell with the nanoparticle composition, whereby the mRNA is delivered to the cell.

In some embodiments of any of the above aspects, a PEG lipid of the nanoparticle composition is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified dialkylglycerol.

In some embodiments, a structural lipid of the nanoparticle composition is selected from the group consisting of cholesterol, fecosterol, sitosterol, campesterol, brassicasterol, stigmasterol, ergosterol, tomatidine, ursolic acid, and alpha-tocopherol. In certain embodiments, the structural lipid is cholesterol.

In some embodiments, a phospholipid of the nanoparticle composition includes a phospholipid moiety and one or more fatty acid moieties, one or more of which may be unsaturated. For example, a nanoparticle composition of the invention may include a lipid according to formula (II)

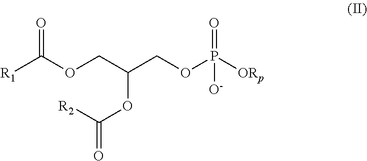

in which Rₚ represents a phospholipid moiety and R₁ and R₂ represent unsaturated fatty acid moieties that may be the same or different. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid. For example, in particular embodiments, a phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine,1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In particular embodiments, the phospholipid is DOPE. Non-natural species including natural species with modifications and substitutions including branching, oxidation, cyclization, and alkynes are also contemplated.

In some embodiments, the lipid component of the nanoparticle composition includes about 35 mol % to about 45 mol % compound of formula (I), about 10 mol % to about 20 mol % DOPE, about 38.5 mol % to about 48.5 mol % structural lipid, and about 1.5 mol % PEG lipid. In a particular embodiment, the lipid component includes about 40 mol % said compound, about 20 mol % DOPE, about 38.5 mol % structural lipid, and about 1.5 mol % PEG lipid.

In other embodiments, the lipid component includes about 40 mol % compound of formula (I), about 15 mol % phospholipid, about 43.5 mol % structural lipid, and about 1.5 mol % PEG lipid. In further embodiments, the lipid component includes about 45 mol % to about 55 mol % compound of formula (I), about 15 mol % to about 25 mol % phospholipid, about 23.5 mol % to about 33.5 mol % structural lipid, and about 1.5 mol % PEG lipid. In particular embodiments, the lipid component includes about 50 mol % said compound, about 20 mol % phospholipid, about 28.5 mol % structural lipid, and about 1.5 mol % PEG lipid. In some of these embodiments, the phospholipid is DOPE, while in other embodiments the phospholipid is DSPC. In certain embodiments, the structural lipid is cholesterol.

In some embodiments, the nanoparticle composition includes more than one phospholipid, PEG lipid, structural lipid, or other lipid. In particular embodiments, the nanoparticle composition further includes a cationic and/or ionizable lipid such as an aminolipid. In certain embodiments, a cationic and/or ionizable lipid is selected from the group consisting of KL10, KL25, 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA or MC3), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyi}oxy)-N,N-dimethyl-3-[(9Z,12Z)-octadeca-9,12-dien-1-yloxy]pro pan-1-amine (Octyi-CLinDMA (2R)), and (2S)-2-({8-[(3β)-cholest-5-en-3-yloxy]octyl}oxy)-N,N-di methyl-3-[(9Z,12Z)-octadeca-9,12-dien-1-yloxy]propan-1-amine (Octyl-CLinDMA (2S)).

In certain embodiments, the nanoparticle composition includes more than one mRNA. An mRNA of a nanoparticle composition of the invention may include one or more of a stem loop, a chain terminating nucleoside, a polyA sequence, a polyadenylation signal, and/or a 5′ cap structure.

In some embodiments, the nanoparticle composition includes more than one mRNA. In certain embodiments, one or more nanoparticle compositions each including one or more mRNAs may be combined and/or simultaneously contacted with a cell.

In some embodiments, the nanoparticle composition includes one or more other components, including, but not limited to, one or more pharmaceutically acceptable excipients, small hydrophobic molecules, therapeutic agents, carbohydrates, polymers, permeability enhancing molecules, and surface altering agents.

In some embodiments, the wt/wt ratio of the lipid component to the mRNA in the nanoparticle composition is from about 5:1 to about 50:1. In certain embodiments, the wt/wt ratio is from about 10:1 to about 40:1.

In some embodiments, the N:P ratio of the nanoparticle composition is from about 2:1 to about 8:1. In particular embodiments, the N:P ratio is from about 2:1 to about 5:1. In preferred embodiments, the N:P ratio is about 4:1. In certain embodiments, the N:P ratio is from about 5:1 to about 8:1. For example, the N:P ratio may be about 5.0:1, about 5.5:1, about 5.67:1, about 6.0:1, about 6.5:1, or about 7.0:1.

In some embodiments, the mean size of the nanoparticle composition is from about 40 nm to about 150 nm. In certain embodiments, the mean size is from about 80 nm to about 120 nm. In one embodiment, the mean size is about 90 nm.

The polydispersity index of the nanoparticle composition is from about 0 to about 0.18 in certain embodiments. In particular embodiments, the polydispersity index is from about 0.13 to about 0.17.

In some embodiments, the nanoparticle composition has a zeta potential of about −10 mV to about +20 mV.

In some embodiments, the encapsulation efficiency of an mRNA of a nanoparticle composition is at least 50%. In particular embodiments, the encapsulation efficiency is at least 80%. In certain embodiments, the encapsulation efficiency is at least 90%.

In certain embodiments of the above methods, the mammalian cell contacted in a method of the invention is in a mammal. In particular embodiments, the nanoparticle composition is administered intravenously, intramuscularly, intradermally, or subcutaneously. A dose of about 0.005 mg/kg to about 5 mg/kg is administered to a mammal in particular embodiments.

### BRIEF DESCRIPTION OF THE DRAWINGS

FIG. 1 displays the encapsulation efficiency and size for nanoparticle compositions of the inventions with different N:P ratios.

FIG. 2 shows the protein expression in mice administered nanoparticle compositions of the invention with different N:P ratios.

FIGS. 3A and 3B demonstrate the encapsulation efficiency and size, respectively, of nanoparticle compositions of the invention including varying relative amounts of KL22.

FIGS. 4A and 4B demonstrate the encapsulation efficiency and size, respectively, of nanoparticle compositions of the invention including varying relative amounts of either DOPE or DSPC.

FIGS. 5A and 5B demonstrate the encapsulation efficiency and size, respectively, of nanoparticle compositions of the invention including varying relative amounts of cholesterol.

FIG. 6 shows the total bioluminescent flux in photons per second at various time points in mice administered particular formulations of nanoparticle compositions of the invention.

FIGS. 7A and 7B show the protein expression in mice intramuscularly (FIG. 7A) or intravenously (FIG. 7B) injected with nanoparticle compositions including various cationic lipids.

FIGS. 8A and 8B show the protein expression in wild type and low density lipoprotein receptor (LDLR) deficient mice intramuscularly (FIG. 8A) or intravenously (FIG. 8B) injected with nanoparticle compositions including KL22 or MC3.

FIGS. 9A and 9B show the protein expression in wild type and apolipoprotein E (apoE) deficient mice intramuscularly (FIG. 9A) or intravenously (FIG. 9B) injected with nanoparticle compositions including KL22 or MC3.

FIG. 10 displays the protein expression in mice administered a single 0.5 mg/kg dose of a nanoparticle composition including KL22 or MC3.

FIG. 11 shows the protein expression in mice administered varying doses of nanoparticle compositions including KL22, MC3, or C12-200.

FIGS. 12A-12G show the levels of TNF-alpha, IFN-gamma, IP-10, MCP-1, IFN-alpha, IL-6, and IL-5 cytokines in mice induced by administration of nanoparticle compositions including KL22, C12-200, or MC3.

### DETAILED DESCRIPTION

This invention relates to nanoparticle compositions including an mRNA and a lipid component and methods of using the same. For example, the invention provides a method of producing a polypeptide of interest in a cell that involves contacting a nanoparticle composition of the invention with a mammalian cell, whereby the mRNA may be translated to produce the polypeptide of interest. The invention further includes a method of delivering an mRNA to a mammalian cell involving administration of a nanoparticle composition including mRNA to a subject, in which the administration involves contacting a cell with the composition, whereby the mRNA is delivered to a cell.

Nanoparticle compositions of the invention comprise an mRNA and a lipid component. A lipid component includes a compound according to formula (I)

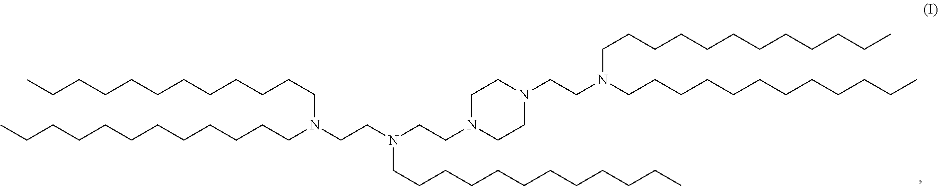 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

a phospholipid (such as a (poly)unsaturated lipid), a structural lipid, and a PEG lipid.

### RNA

An RNA may be a messenger RNA (mRNA). An mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine.

A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component.

A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analog thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analog or derivative thereof and may include one or more substitutions or modifications as described herein. For example, “guanosine triphosphate” is understood to include the canonical guanosine triphosphate, 7-methylguanosine triphosphate, or any other definition encompassed herein.

An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified. For example, all cytosine in an mRNA may be 5-methylcytosine.

In some embodiments, an mRNA may include a 5′ cap structure, a chain terminating nucleotide, a stem loop, a polyA sequence, and/or a polyadenylation signal.

A cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. Cap species include m⁷GpppG, m⁷Gpppm⁷G, m⁷3′dGpppG, m₂^{7,O3}′GpppG, m₂^{7,O3}′GppppG, m₂^{7,O2}′GppppG, m⁷Gpppm⁷G, m⁷3′dGpppG, m₂^{7,O3}′GpppG, m₂^{7,O3}′GppppG, and m₂^{7,O2}′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine.

An mRNA may instead or additionally include a stem loop, such as a histone stem loop. A stem loop may include 1, 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA.

An mRNA may encode any polypeptide of interest, including any naturally or non-naturally occurring or otherwise modified polypeptide. A polypeptide encoded by an mRNA may be of any size and may have any secondary structure or activity. In some embodiments, a polypeptide encoded by an mRNA may have a therapeutic effect when expressed in a cell.

### KL22 and Cationic/Ionizable Lipids

Nanoparticle compositions of the invention comprise a lipid component in addition to mRNA. The lipid component of a nanoparticle composition may include one or more lipids, including a compound according to formula (I). The compound according to formula (I) is also referred to herein as KL22.

KL22 may be prepared via a reductive amination reaction between a carbonyl and a polyamine. For example, the polyamine N-{2-[4-(2-aminoethyl)piperazin-1-yl]ethyl}-1,2-ethanediamine may be reacted with the aldehyde dodecanal in the presence of a reducing agent to produce KL22. A reducing agent is typically a species that donates electronic character to another species during an oxidation-reduction reaction. In the present reaction, a reducing agent is a species capable of reducing an imine intermediate produced in a reaction between an amine and a carbonyl. Such species are well known in the chemical arts and may be selected from, for example, sodium cyanoborohydride and sodium triacetoxyborohydride,

At least one nitrogen atom of KL22 may be protonated at a physiological pH. Thus, KL22 may have a positive or partial positive charge at physiological pH. KL22 may be referred to as a cationic or ionizable (amino)lipid.

In addition to KL22, a nanoparticle composition may include one or more additional lipids. For example, a nanoparticle composition may include one or more cationic and/or ionizable lipids. Cationic and/or ionizable lipids may be selected from the non-limiting group consisting of 3-(didodecylamino)-N1,N1,4-tridodecyl-1-piperazineethanamine (KL10), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA),1,2-dioleyloxy-N,N-dimethylaminopropane (DODMA), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]pro pan-1-amine (Octyl-CLinDMA (2R)), and (2S)-2-({8-[(3β)-cholest-5-en-3-yloxy]octyl}oxy)-N, N-di methyl-3-[(9Z,12Z)-octadeca-9,12-dien-1-yloxy]prop an-1-amine (Octyl-CLinDMA (2S)). In addition to these, a cationic lipid may also be a lipid including a cyclic amine.

### PEG Lipids

The lipid component of a nanoparticle composition of the invention may include one or more PEG or PEG-modified lipids. Such species may be alternately referred to as PEGylated lipids. A PEG lipid is a lipid modified with polyethylene glycol.

The lipid component may include one or more PEG lipids. A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEG lipid may be PEG-c-DOMG, PEG-DMG, PEG-DLPE, PEG-DMPE, PEG-DPPC, or a PEG-DSPE lipid.

### Structural Lipids

The lipid component of a nanoparticle composition may include one or more structural lipids. The nanoparticle compositions of the present invention may include a structural lipid (e.g., cholesterol fecosterol, sitosterol, campesterol, stigmasterol, brassicasterol, ergosterol, tomatidine, tomatine, ursolic acid, or alpha-tocopherol).

### Phospholipids

The lipid component of a nanoparticle composition may include one or more phospholipids, such as one or more (poly)unsaturated lipids. In general, such lipids may include a phospholipid moiety and one or more fatty acid moieties. For example, a phospholipid may be a lipid according to formula (II)

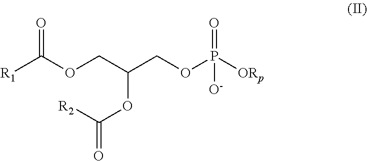

in which Rₚ represents a phospholipid moiety and R₁ and R₂ represent fatty acid moieties with or without saturation that may be the same or different. A phospholipid moiety may be selected from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin. A fatty acid moiety may be selected from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid. Non-natural species including natural species with modifications and substitutions including branching, oxidation, cyclization, and alkynes are also contemplated.

In some embodiments a nanoparticle composition may include 1,2-distearoyl-sn-glycero-3-phosphocholine (DSPC), 1,2-dioleoyl-sn-glycero-3-phosphoethanolamine (DOPE), or both DSPC and DOPE. Phospholipids useful in the compositions and methods of the invention may be selected from the non-limiting group consisting of DSPC, DOPE, 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

### Other Components

A nanoparticle composition may include one or more components in addition to those described in the preceding sections. For example, a nanoparticle composition may include one or more small hydrophobic molecules such as a vitamin (e.g., vitamin A or vitamin E) or a sterol.

Nanoparticle compositions may also include one or more permeability enhancer molecules, carbohydrates, polymers, therapeutic agents, surface altering agents, or other components. A permeability enhancer molecule may be a molecule described by U.S. patent application publication No. 2005/0222064, for example. Carbohydrates may include simple sugars (e.g., glucose) and polysaccharides (e.g., glycogen and derivatives and analogs thereof).

A polymer may be included in and/or used to encapsulate or partially encapsulate a nanoparticle composition. A polymer may be biodegradable and/or biocompatible. A polymer may be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. For example, a polymer may include poly(caprolactone) (PCL), ethylene vinyl acetate polymer (EVA), poly(lactic acid) (PLA), poly(L-lactic acid) (PLLA), poly(glycolic acid) (PGA), poly(lactic acid-co-glycolic acid) (PLGA), poly(L-lactic acid-co-glycolic acid) (PLLGA), poly(D,L-lactide) (PDLA), poly(L-lactide) (PLLA), poly(D,L-lactide-co-caprolactone), poly(D,L-lactide-co-caprolactone-co-glycolide), poly(D,L-lactide-co-PEO-co-D,L-lactide), poly(D,L-lactide-co-PPO-co-D,L-lactide), polyalkyl cyanoacralate, polyurethane, poly-L-lysine (PLL), hydroxypropyl methacrylate (HPMA), polyethyleneglycol, poly-L-glutamic acid, poly(hydroxy acids), polyanhydrides, polyorthoesters, poly(ester amides), polyamides, poly(ester ethers), polycarbonates, polyalkylenes such as polyethylene and polypropylene, polyalkylene glycols such as poly(ethylene glycol) (PEG), polyalkylene oxides (PEO), polyalkylene terephthalates such as poly(ethylene terephthalate), polyvinyl alcohols (PVA), polyvinyl ethers, polyvinyl esters such as poly(vinyl acetate), polyvinyl halides such as poly(vinyl chloride) (PVC), polyvinylpyrrolidone, polysiloxanes, polystyrene (PS), polyurethanes, derivatized celluloses such as alkyl celluloses, hydroxyalkyl celluloses, cellulose ethers, cellulose esters, nitro celluloses, hydroxypropylcellu lose, carboxymethylcellulose, polymers of acrylic acids, such as poly(methyl(meth)acrylate) (PMMA), poly(ethyl(meth)acrylate), poly(butyl(meth)acrylate), poly(isobutyl(meth)acrylate), poly(hexyl(meth)acrylate), poly(isodecyl(meth)acrylate), poly(lauryl(meth)acrylate), poly(phenyl(meth)acrylate), poly(methyl acrylate), poly(isopropyl acrylate), poly(isobutyl acrylate), poly(octadecyl acrylate) and copolymers and mixtures thereof, polydioxanone and its copolymers, polyhydroxyalkanoates, polypropylene fumarate, polyoxymethylene, poloxamers, polyoxamines, poly(ortho)esters, poly(butyric acid), poly(valeric acid), poly(lactide-co-caprolactone), and trimethylene carbonate, polyvinylpyrrolidone.

Therapeutic agents may include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

Surface altering agents may include, but are not limited to, anionic proteins (e.g., bovine serum albumin), surfactants (e.g., cationic surfactants such as dimethyldioctadecyl-ammonium bromide), sugars or sugar derivatives (e.g., cyclodextrin), nucleic acids, polymers (e.g., heparin, polyethylene glycol, and poloxamer), mucolytic agents (e.g., acetylcysteine, mugwort, bromelain, papain, clerodendrum, bromhexine, carbocisteine, eprazinone, mesna, ambroxol, sobrerol, domiodol, letosteine, stepronin, tiopronin, gelsolin, thymosin 134, dornase alfa, neltenexine, and erdosteine), and DNases (e.g., rhDNase). A surface altering agent may be disposed within a nanoparticle and/or on the surface of a nanoparticle composition (e.g., by coating, adsorption, covalent linkage, or other process).

In addition to these components, nanoparticle compositions of the invention may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro; Lippincott, Williams & Wilkins, Baltimore, Md., 2006).

Examples of diluents may include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and/or combinations thereof. Granulating and dispersing agents may be selected from the non-limiting list consisting of potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, and/or combinations thereof.

Surface active agents and/or emulsifiers may include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN® 60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ® 45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ® 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLURONIC®F 68, POLOXAMER® 188, cetrimonium bromide, cetylpyridinium chloride, benzalkonium chloride, docusate sodium, and/or combinations thereof.

A binding agent may be starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan); alginates; polyethylene oxide; polyethylene glycol; inorganic calcium salts; silicic acid; polymethacrylates; waxes; water; alcohol; and combinations thereof, or any other suitable binding agent.

Preservatives include, but are not limited to, antioxidants, chelating agents, antimicrobial preservatives, antifungal preservatives, alcohol preservatives, acidic preservatives, and/or other preservatives. Antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Examples of alcohol preservatives include, but are not limited to, ethanol, polyethylene glycol, phenol, benzyl alcohol, phenolic compounds, bisphenol, chlorobutanol, hydroxybenzoate, and/or phenylethyl alcohol. Examples of acidic preservatives include, but are not limited to, vitamin A, vitamin C, vitamin E, beta-carotene, citric acid, acetic acid, dehydroascorbic acid, ascorbic acid, sorbic acid, and/or phytic acid. Other preservatives include, but are not limited to, tocopherol, tocopherol acetate, deteroxime mesylate, cetrimide, butylated hydroxyanisole (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL® 115, GERMABEN®II, NEOLONE™, KATHON™, and/or EUXYL®.

Examples of buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, d-gluconic acid, calcium glycerophosphate, calcium lactate, calcium lactobionate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, amino-sulfonate buffers (e.g. HEPES), magnesium hydroxide, aluminum hydroxide, alginic acid, pyrogen-free water, isotonic saline, Ringer's solution, ethyl alcohol, and/or combinations thereof. Lubricating agents may selected from the non-limiting group consisting of magnesium stearate, calcium stearate, stearic acid, silica, talc, malt, glyceryl behenate, hydrogenated vegetable oils, polyethylene glycol, sodium benzoate, sodium acetate, sodium chloride, leucine, magnesium lauryl sulfate, sodium lauryl sulfate, and combinations thereof.

Examples of oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils as well as butyl stearate, caprylic triglyceride, capric triglyceride, cyclomethicone, diethyl sebacate, dimethicone 360, simethicone, isopropyl myristate, mineral oil, octyldodecanol, oleyl alcohol, silicone oil, and/or combinations thereof.

### Compositions

A nanoparticle composition of the invention may include mRNA and a lipid component including one or more lipids. For example, a composition may include mRNA, KL22, a phospholipid (such as an unsaturated lipid, e.g., DOPE), a PEG lipid, and a structural lipid. Examples of formulations of lipid components of nanoparticle compositions are presented in Table 2.

In some embodiments, the lipid component includes KL22, a phospholipid, a PEG lipid, and a structural lipid. The lipid component may include about 35 mol % to about 45 mol % KL22, about 10 mol % to about 20 mol % phospholipid, about 38.5 mol % to about 48.5 mol % structural lipid, and about 1.5 mol % PEG lipid, provided that the total mol % does not exceed 100%. For example, the lipid component may include about 40 mol % KL22, about 20 mol % phospholipid, about 38.5 mol % structural lipid, and about 1.5 mol % PEG lipid. In some embodiments, the phospholipid may be DOPE and/or the structural lipid may be cholesterol.

In some embodiments, the lipid component may include about 40 mol % KL22, about 15 mol % phospholipid, about 43.5 mol % structural lipid, and about 1.5 mol % PEG lipid. In some instances, the phospholipid may be DOPE. In other embodiments, the lipid may be DSPC. In certain embodiments, the structural lipid may be cholesterol.

In other embodiments, the lipid component may include about 45 mol % to about 55 mol % KL22, about 15 mol % to about 25 mol % phospholipid, about 23.5 mol % to about 33.5 mol % structural lipid, and about 1.5 mol % PEG lipid, provided that the total mol % does not exceed 100%. For example, the lipid component may include about 50 mol % KL22, about 20 mol % phospholipid, about 28.5 mol % structural lipid, and about 1.5 mol % PEG lipid. In some embodiments, the phospholipid may be DOPE. In other instances, the phospholipid may be DSPC. In certain embodiments, the structural lipid may be cholesterol.

A nanoparticle composition may be designed for one or more specific applications or targets. For example, a nanoparticle composition may be designed to deliver mRNA to a particular cell, tissue, organ, or system or group thereof in a mammal's body, such as the renal system. Physiochemical properties of nanoparticle compositions may be altered in order to increase selectivity for particular bodily targets. For instance, particle sizes may be adjusted based on the fenestration sizes of different organs. The mRNA included in a nanoparticle composition may also depend on the desired delivery target or targets. For example, an mRNA may be selected for a particular indication, condition, disease, or disorder and/or for delivery to a particular cell, tissue, organ, or system or group thereof (e.g., localized or specific delivery). A nanoparticle composition may include one or more mRNA molecules encoding one or more polypeptides of interest.

The amount of mRNA in a nanoparticle composition may depend on the size, sequence, and other characteristics of the mRNA. The amount of mRNA in a nanoparticle composition may also depend on the size, composition, desired target, and other characteristics of the nanoparticle composition. The relative amounts of mRNA and other elements (e.g., lipids) may also vary. In some embodiments, the wt/wt ratio of the lipid component to an mRNA in a nanoparticle composition may be from about 5:1 to about 50:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, and 50:1. For example, the wt/wt ratio of the lipid component to an mRNA may be from about 10:1 to about 40:1. The amount of mRNA in a nanoparticle composition may, for example, be measured using absorption spectroscopy (e.g., ultraviolet-visible spectroscopy).

In some embodiments, the one or more mRNAs, lipids, and amounts thereof may be selected to provide a specific N:P ratio. The N:P ratio of the composition refers to the molar ratio of nitrogen atoms in one or more lipids to the number of phosphate groups in an mRNA. In general, a lower N:P ratio is preferred. The one or more mRNA, lipids, and amounts thereof may be selected to provide an N:P ratio from about 2:1 to about 8:1, such as 2:1, 3:1, 4:1, 5:1, 6:1, 7:1, and 8:1. In certain embodiments, the N:P ratio may be from about 2:1 to about 5:1. In preferred embodiments, the N:P ratio may be about 4:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

The mean size of a nanoparticle composition of the invention may be between 10 s of nm and 100 s of nm. For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 80 nm to about 120 nm, from about 80 nm to about 110 nm, from about 80 nm to about 100 nm, from about 80 nm to about 90 nm, from about 90 nm to about 120 nm, from about 90 nm to about 110 nm, from about 90 nm to about 100 nm, from about 100 nm to about 120 nm, or from about 110 nm to about 120 nm. In a particular embodiment, the mean size may be about 90 nm. In another particular embodiment, the mean size may be about 100 nm.

A nanoparticle composition of the invention may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition of the invention may have a polydispersity index from about 0 to about 0.18, such as 0.01, 0.02, 0.03, 0.04, 0.05, 0.06, 0.07, 0.08, 0.09, 0.10, 0.11, 0.12, 0.13, 0.14, 0.15, 0.16, 0.17, or 0.18. In some embodiments, the polydispersity index of a nanoparticle composition may be from about 0.13 to about 0.17.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition of the invention may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of an mRNA describes the amount of mRNA that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of mRNA in a solution containing the nanoparticle composition before and after breaking up the nanoparticle composition with one or more organic solvents or detergents. Fluorescence may be used to measure the amount of free mRNA in a solution. For the nanoparticle compositions of the invention, the encapsulation efficiency of an mRNA may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition of the invention may optionally comprise one or more coatings. For example, a nanoparticle composition may be formulated in a capsule, film, or tablet having a coating. A capsule, film, or tablet including a composition of the invention may have any useful size, tensile strength, hardness, or density.

### Pharmaceutical Compositions

Nanoparticle compositions of the invention may be formulated in whole or in part as pharmaceutical compositions. Pharmaceutical compositions of the invention may include one or more nanoparticle compositions. For example, a pharmaceutical composition may include one or more nanoparticle compositions including one or more different mRNAs. Pharmaceutical compositions of the invention may further include one or more pharmaceutically acceptable excipients or accessory ingredients such as those described herein. General guidelines for the formulation and manufacture of pharmaceutical compositions and agents are available, for example, in Remington's The Science and Practice of Pharmacy, 21^{st} Edition, A. R. Gennaro; Lippincott, Williams & Wilkins, Baltimore, Md., 2006. Conventional excipients and accessory ingredients may be used in any pharmaceutical composition of the invention, except insofar as any conventional excipient or accessory ingredient may be incompatible with one or more components of a nanoparticle composition of the invention. An excipient or accessory ingredient may be incompatible with a component of a nanoparticle composition if its combination with the component may result in any undesirable biological effect or otherwise deleterious effect.

In some embodiments, one or more excipients or accessory ingredients may make up greater than 50% of the total mass or volume of a pharmaceutical composition including a nanoparticle composition of the invention. For example, the one or more excipients or accessory ingredients may make up 50%, 60%, 70%, 80%, 90%, or more of a pharmaceutical convention. In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of the one or more nanoparticle compositions, the one or more pharmaceutically acceptable excipients, and/or any additional ingredients in a pharmaceutical composition in accordance with the present disclosure will vary, depending upon the identity, size, and/or condition of the subject treated and further depending upon the route by which the composition is to be administered. By way of example, a pharmaceutical composition may comprise between 0.1% and 100% (wt/wt) of one or more nanoparticle compositions.

Nanoparticle compositions and/or pharmaceutical compositions including one or more nanoparticle compositions may be administered to any patient or subject, including those patients or subjects that may benefit from a therapeutic effect provided by the delivery of an mRNA to one or more particular cells, tissues, organs, or systems or groups thereof, such as the renal system. Although the descriptions provided herein of nanoparticle compositions and pharmaceutical compositions including nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the compositions is contemplated include, but are not limited to, humans, other primates, and other mammals, including commercially relevant mammals such as cattle, pigs, hoses, sheep, cats, dogs, mice, and/or rats.

A pharmaceutical composition including one or more nanoparticle compositions may be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., nanoparticle composition). The amount of the active ingredient is generally equal to the dosage of the active ingredient which would be administered to a subject and/or a convenient fraction of such a dosage such as, for example, one-half or one-third of such a dosage.

Pharmaceutical compositions of the invention may be prepared in a variety of forms suitable for a variety of routes and methods of administration. For example, pharmaceutical compositions of the invention may be prepared in liquid dosage forms (e.g., emulsions, microemulsions, nanoemulsions, solutions, suspensions, syrups, and elixirs), injectable forms, solid dosage forms (e.g., capsules, tablets, pills, powders, and granules), dosage forms for topical and/or transdermal administration (e.g., ointments, pastes, creams, lotions, gels, powders, solutions, sprays, inhalants, and patches), suspensions, powders, and other forms.

Liquid dosage forms for oral and parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are prepared by entrapping the drug in liposomes or microemulsions which are compatible with body tissues.

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, films, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay, silicates), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

Dosage forms for topical and/or transdermal administration of a composition may include ointments, pastes, creams, lotions, gels, powders, solutions, sprays, inhalants, and/or patches. Generally, an active ingredient is admixed under sterile conditions with a pharmaceutically acceptable excipient and/or any needed preservatives and/or buffers as may be required. Additionally, the present disclosure contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662. Intradermal compositions may be administered by devices which limit the effective penetration length of a needle into the skin, such as those described in PCT publication WO 99/34850 and functional equivalents thereof. Jet injection devices which deliver liquid compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate vaccine in powder form through the outer layers of the skin to the dermis are suitable. Alternatively or additionally, conventional syringes may be used in the classical mantoux method of intradermal administration.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (wt/wt) active ingredient, although the concentration of active ingredient may be as high as the solubility limit of the active ingredient in the solvent. Formulations for topical administration may further comprise one or more of the additional ingredients described herein.

A pharmaceutical composition may be prepared, packaged, and/or sold in a formulation suitable for pulmonary administration via the buccal cavity. Such a formulation may comprise dry particles which comprise the active ingredient and which have a diameter in the range from about 0.5 nm to about 7 nm or from about 1 nm to about 6 nm. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (wt/wt) of the composition, and active ingredient may constitute 0.1% to 20% (wt/wt) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 1 nm to about 200 nm.

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (wt/wt) and as much as 100% (wt/wt) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, 0.1% to 20% (wt/wt) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

A pharmaceutical composition may be prepared, packaged, and/or sold in a formulation suitable for ophthalmic administration. Such formulations may, for example, be in the form of eye drops including, for example, a 0.1/1.0% (wt/wt) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this present disclosure.

### Methods of Producing Polypeptides in Cells

The present disclosure provides methods of producing a polypeptide of interest in a mammalian cell. Methods of producing polypeptides involve contacting a cell with a nanoparticle composition including an mRNA encoding the polypeptide of interest. Upon contacting the cell with the nanoparticle composition, the mRNA may be taken up and translated in the cell to produce the polypeptide of interest.

In general, the step of contacting a mammalian cell with a nanoparticle composition including an mRNA encoding a polypeptide of interest may be performed in vivo, ex vivo, in culture, or in vitro. The amount of nanoparticle composition contacted with a cell, and/or the amount of mRNA therein, may depend on the type of cell or tissue being contacted, the means of administration, the physiochemical characteristics of the nanoparticle composition and the mRNA (e.g., size, charge, and chemical composition) therein, and other factors. In general, an effective amount of the nanoparticle composition will allow for efficient polypeptide production in the cell. Metrics for efficiency may include polypeptide translation (indicated by polypeptide expression), level of mRNA degradation, and immune response indicators.

The step of contacting a nanoparticle composition including an mRNA with a cell may involve or cause transfection. A phospholipid including in the lipid component of a nanoparticle composition may facilitate transfection and/or increase transfection efficiency, for example, by interacting and/or fusing with a cellular or intracellular membrane. Transfection may allow for the translation of the mRNA within the cell.

In some embodiments, the nanoparticle compositions described herein may be used as therapeutic agents. For example, an mRNA included in a nanoparticle composition may encode a therapeutic polypeptide (e.g., in a translatable region) and produce the therapeutic polypeptide upon contacting and/or entry (e.g., transfection) into a cell. In other embodiments, an mRNA included in a nanoparticle composition of the invention may encode a polypeptide that may improve or increase the immunity of a subject. For example, an mRNA may encode a granulocyte-colony stimulating factor or trastuzumab.

In certain embodiments, an mRNA included in a nanoparticle composition of the invention may encode a recombinant polypeptide that may replace one or more polypeptides that may be substantially absent in a cell contacted with the nanoparticle composition. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the cell. Antagonized biological moieties may include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the cell, such as within a specific compartment such as the nucleus, or may be engineered for secretion from the cell or for translocation to the plasma membrane of the cell.

In some embodiments, contacting a cell with a nanoparticle composition including an mRNA may reduce the innate immune response of a cell to an exogenous nucleic acid. A cell may be contacted with a first nanoparticle composition including a first amount of a first exogenous mRNA including a translatable region and the level of the innate immune response of the cell to the first exogenous mRNA may be determined. Subsequently, the cell may be contacted with a second composition including a second amount of the first exogenous mRNA, the second amount being a lesser amount of the first exogenous mRNA compared to the first amount. Alternatively, the second composition may include a first amount of a second exogenous mRNA that is different from the first exogenous mRNA. The steps of contacting the cell with the first and second compositions may be repeated one or more times. Additionally, efficiency of polypeptide production (e.g., translation) in the cell may be optionally determined, and the cell may be re-contacted with the first and/or second composition repeatedly until a target protein production efficiency is achieved.

Methods of Delivering mRNA to Cells

The present disclosure provides methods of delivering an mRNA to a mammalian cell. Delivery of an mRNA to a cell involves administering a nanoparticle composition including the mRNA to a subject, where administration of the composition involves contacting the cell with the composition. Upon contacting the cell with the nanoparticle composition, a translatable mRNA may be translated in the cell to produce a polypeptide of interest. However, mRNAs that are substantially not translatable may also be delivered to cells. Substantially non-translatable mRNAs may be useful as vaccines and/or may sequester translational components of a cell to reduce expression of other species in the cell.

In some embodiments, a nanoparticle composition of the invention may target a particular type or class of cells. For example, an mRNA that encodes a protein-binding partner (e.g., an antibody or functional fragment thereof, a scaffold protein, or a peptide) or a receptor on a cell surface may be included in a nanoparticle composition. An mRNA may additionally or instead be used to direct the synthesis and extracellular localization of lipids, carbohydrates, or other biological moieties. Alternatively, other elements (e.g., lipids or ligands) of a nanoparticle composition may be selected based on their affinity for particular receptors (e.g., low density lipoprotein receptors) such that a nanoparticle composition may more readily interact with a target cell population including the receptors. For example, ligands may include, but are not limited to, members of a specific binding pair, antibodies, monoclonal antibodies, Fv fragments, single chain Fv (scFv) fragments, Fab′ fragments, F(ab′)2 fragments, single domain antibodies, camelized antibodies and fragments thereof, humanized antibodies and fragments thereof, and multivalent versions thereof; multivalent binding reagents including mono- or bi-specific antibodies such as disulfide stabilized Fv fragments, scFv tandems, diabodies, tridobdies, or tetrabodies; and aptamers, receptors, and fusion proteins.

In some embodiments, a ligand may be a surface-bound antibody, which can permit tuning of cell targeting specificity. This is especially useful since highly specific antibodies can be raised against an epitope of interest for the desired targeting site. In one embodiment, multiple antibodies are expressed on the surface of a cell, and each antibody can have a different specificity for a desired target. Such approaches can increase the avidity and specificity of targeting interactions.

A ligand can be selected, e.g., by a person skilled in the biological arts, based on the desired localization or function of the cell. For example an estrogen receptor ligand, such as tamoxifen, can target cells to estrogen-dependent breast cancer cells that have an increased number of estrogen receptors on the cell surface. Other non-limiting examples of ligand/receptor interactions include CCR1 (e.g., for treatment of inflamed joint tissues or brain in rheumatoid arthritis, and/or multiple sclerosis), CCR7, CCR8 (e.g., targeting to lymph node tissue), CCR6, CCR9,CCR10 (e.g., to target to intestinal tissue), CCR4, CCR10 (e.g., for targeting to skin), CXCR4 (e.g., for general enhanced transmigration), HCELL (e.g., for treatment of inflammation and inflammatory disorders, bone marrow), Alpha4beta7 (e.g., for intestinal mucosa targeting), and VLA-4NCAM-1 (e.g., targeting to endothelium). In general, any receptor involved in targeting (e.g., cancer metastasis) can be harnessed for use in the methods and compositions described herein.

Targeted cells may include, but are not limited to, hepatocytes, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells.

In particular embodiments, a nanoparticle composition of the invention may target hepatocytes. Apolipoprotiens such as apolipoprotein E (apoE) have been shown to associate with neutral or near neutral lipid-containing nanoparticle compositions in the body, and are known to associate with receptors such as low-density lipoprotein receptors (LDLRs) found on the surface of hepatocytes. Thus, a nanoparticle composition including a lipid component with a neutral or near neutral charge that is administered to a subject may acquire apoE in a subject's body and may subsequently deliver mRNA to hepatocytes including LDLRs in a targeted manner.

Nanoparticle compositions of the invention may be useful for treating a disease, disorder, or condition characterized by missing or aberrant protein or polypeptide activity. Upon delivery of an mRNA encoding the missing or aberrant polypeptide to a cell, translation of the mRNA may produce the polypeptide, thereby reducing or eliminating an issue caused by the absence of or aberrant activity caused by the polypeptide. Because translation may occur rapidly, the methods and compositions of the invention may be useful in the treatment of acute diseases, disorders, or conditions such as sepsis, stroke, and myocardial infarction. An mRNA included in a nanoparticle composition of the invention may also be capable of altering the rate of transcription of a given species, thereby affecting gene expression.

Diseases, disorders, and/or conditions characterized by dysfunctional or aberrant protein or polypeptide activity for which a composition of the invention may be administered include, but are not limited to, cancer and proliferative diseases, genetic diseases (e.g., cystic fibrosis), autoimmune diseases, diabetes, neurodegenerative diseases, cardio- and reno-vascular diseases, and metabolic diseases. Multiple diseases, disorders, and/or conditions may be characterized by missing (or substantially diminished such that proper protein function does not occur) protein activity. Such proteins may not be present, or they may be essentially non-functional. A specific example of a dysfunctional protein is the missense mutation variants of the cystic fibrosis transmembrane conductance regulator (CFTR) gene, which produce a dysfunctional protein variant of CFTR protein, which causes cystic fibrosis. The present disclosure provides a method for treating such diseases, disorders, and/or conditions in a subject by administering a nanoparticle composition including an mRNA and a lipid component including KL22, a phospholipid (optionally unsaturated), a PEG lipid, and a structural lipid, wherein the mRNA encodes a polypeptide that antagonizes or otherwise overcomes an aberrant protein activity present in the cell of the subject.

The invention provides methods involving administering nanoparticle compositions including mRNA or pharmaceutical compositions including the same. Compositions of the invention, or imaging, diagnostic, or prophylactic compositions thereof, may be administered to a subject using any reasonable amount and any route of administration effective for preventing, treating, diagnosing, or imaging a disease, disorder, and/or condition and/or any other purpose. The specific amount administered to a given subject may vary depending on the species, age, and general condition of the subject; the purpose of the administration; the particular composition; the mode of administration; and the like. Compositions in accordance with the present disclosure may be formulated in dosage unit form for ease of administration and uniformity of dosage. It will be understood, however, that the total daily usage of a composition of the present disclosure will be decided by an attending physician within the scope of sound medical judgment. The specific therapeutically effective, prophylactically effective, or otherwise appropriate dose level (e.g., for imaging) for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

A nanoparticle composition including one or more mRNAs may be administered by any route. In some embodiments, compositions of the invention, including prophylactic, diagnostic, or imaging compositions including one or more nanoparticle compositions of the invention, are administered by one or more of a variety of routes, including oral, intravenous, intramuscular, intra-arterial, intramedullary, intrathecal, subcutaneous, intraventricular, trans- or intra-dermal, interdermal, rectal, intravaginal, intraperitoneal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, or subcutaneously. However, the present disclosure encompasses the delivery of compositions of the invention by any appropriate route taking into consideration likely advances in the sciences of drug delivery. In general, the most appropriate route of administration will depend upon a variety of factors including the nature of the nanoparticle composition including one or more mRNAs (e.g., its stability in various bodily environments such as the bloodstream and gastrointestinal tract), the condition of the patient (e.g., whether the patient is able to tolerate particular routes of administration), etc.

In certain embodiments, compositions in accordance with the present disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of a composition per 1 kg of subject body weight. In particular embodiments, a dose of about 0.005 mg/kg to about 5 mg/kg of a nanoparticle composition of the invention may be administrated. A dose may be administered one or more times per day, in the same or a different amount, to obtain a desired level of mRNA expression and/or therapeutic, diagnostic, prophylactic, or imaging effect. The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition.

Nanoparticle compositions including one or more mRNAs may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. For example, one or more nanoparticle compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

It will further be appreciated that therapeutically, prophylactically, diagnostically, or imaging active agents utilized in combination may be administered together in a single composition or administered separately in different compositions. In general, it is expected that agents utilized in combination will be utilized at levels that do not exceed the levels at which they are utilized individually. In some embodiments, the levels utilized in combination may be lower than those utilized individually.

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### Definitions

About, Approximately: As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a lipid component of a nanoparticle composition, “about” may mean+/−10% of the recited value. For instance, a nanoparticle composition including a lipid component having about 40% of a given compound may include 30-50% of the compound.

Composition/nanoparticle composition: As used herein, “composition” or “nanoparticle composition” refers to a mixture or formulation that includes an mRNA and a lipid component.

Compound: As used herein, the term “compound,” is meant to include all geometric isomers and isotopes of the structure depicted. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Contacting: As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

Delivering: As used herein, the term “delivering” means providing an entity to a destination. For example, delivering an mRNA to a subject may involve administering a nanoparticle composition including the mRNA to the subject (e.g., by an intravenous, intramuscular, intradermal, or subcutaneous route). Administration of a nanoparticle composition to a mammal or mammalian cell may involve contacting one or more cells with the nanoparticle composition.

Single unit dose: As used herein, a “single unit dose” is a dose of any therapeutic administered in one dose/at one time/single route/single point of contact, i.e., single administration event.

Split dose: As used herein, a “split dose” is the division of single unit dose or total daily dose into two or more doses.

Total daily dose: As used herein, a “total daily dose” is an amount given or prescribed in 24 hour period. It may be administered as a single unit dose.

Encapsulation efficiency: As used herein, “encapsulation efficiency” refers to the amount of an mRNA that becomes part of a nanoparticle composition, relative to the initial total amount of mRNA used in the preparation of a nanoparticle composition. For example, if 97 mg of mRNA are encapsulated in a nanoparticle composition out of a total 100 mg of mRNA initially provided to the composition, the encapsulation efficiency may be given as 97%. As used herein, “encapsulation” may refer to complete, substantial, or partial enclosure, confinement, surrounding, or encasement.

Expression: As used herein, “expression” of a nucleic acid sequence refers to translation of an mRNA into a polypeptide or protein and/or post-translational modification of a polypeptide or protein.

Phospholipid: As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Ex vivo: As used herein, the term “ex vivo” refers to events that occur outside of an organism (e.g., animal, plant, or microbe or cell or tissue thereof). Ex vivo events may take place in an environment minimally altered from a natural (e.g., in vivo) environment.

Linker: As used herein, a “linker” is a moiety connecting two moieties, for example, the connection between two nucleosides of a cap species. A linker may include one or more groups including but not limited to phosphate groups (e.g., phosphates, boranophosphates, thiophosphates, selenophosphates, and phosphonates), alkyl groups, amidates, or glycerols. For example, two nucleosides of a cap analog may be linked at their 5′ positions by a triphosphate group or by a chain including two phosphate moieties and a boranophosphate moiety.

Lipid component: As used herein, a “lipid component” is that component of a nanoparticle composition that includes one or more lipids. For example, the lipid component may include one or more cationic/ionizable, PEGylated, structural, or other lipids, such as phospholipids.

Methods of administration: As used herein, “methods of administration” may include intravenous, intramuscular, intradermal, subcutaneous, or other methods of delivering a composition to a subject. A method of administration may be selected to target delivery to a specific region or system of a body.

Modified: As used herein, “modified” means non-natural. For example, an mRNA may be a modified mRNA. That is, an mRNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

mRNA: As used herein, an “mRNA” refers to a messenger ribonucleic acid that may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide of interest. Translation of an mRNA, for example, in vivo translation of an mRNA inside a mammalian cell, may produce a polypeptide of interest.

N:P ratio: As used herein, the “N:P ratio” is the molar ratio of ionizable (in the physiological pH range) nitrogen atoms in a lipid to phosphate groups in an RNA, e.g., in a nanoparticle composition including a lipid component and an RNA, such as an mRNA.

Naturally occurring: As used herein, “naturally occurring” means existing in nature without artificial aid.

Patient: As used herein, “patient” refers to a subject who may seek or be in need of treatment, requires treatment, is receiving treatment, will receive treatment, or a subject who is under care by a trained professional for a particular disease or condition.

PEG lipid: As used herein, a “PEG lipid” or “PEGylated lipid” refers to a lipid comprising a polyethylene glycol component.

Pharmaceutically acceptable: The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipient: The phrase “pharmaceutically acceptable excipient,” as used herein, refers to any ingredient other than the compounds described herein (for example, a vehicle capable of suspending, complexing, or dissolving the active compound) and having the properties of being substantially nontoxic and non-inflammatory in a patient. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E (alpha-tocopherol), vitamin C, xylitol, and other species disclosed herein.

Pharmaceutically acceptable salts: Compositions of the invention may also include pharmaceutically acceptable salts of one or more compounds. As used herein, “pharmaceutically acceptable salts” refers to derivatives of the disclosed compounds wherein the parent compound is altered by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polydispersity index: As used herein, the “polydispersity index” is a ratio that describes the homogeneity of the particle size distribution of a system. A small value, e.g., less than 0.3, indicates a narrow particle size distribution.

Polypeptide: As used herein, the term “polypeptide” or “polypeptide of interest” refers to a polymer of amino acid residues typically joined by peptide bonds that can be produced naturally (e.g., isolated or purified) or synthetically.

Size: As used herein, “size” or “mean size” in the context of nanoparticle compositions refers to the mean diameter of a nanoparticle composition.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Targeted cells: As used herein, “targeted cells” refers to any one or more cells of interest. The cells may be found in vitro, in vivo, in situ or in the tissue or organ of an organism. The organism may be an animal, preferably a mammal, more preferably a human and most preferably a patient.

Therapeutic agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, composition, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Transfection: As used herein, “transfection” refers to the introduction of a species (e.g., an mRNA) into a cell. Transfection may occur, for example, in vitro, ex vivo, or in vivo.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Zeta potential: As used herein, the “zeta potential” is the electrokinetic potential of a lipid e.g., in a particle composition.

### EQUIVALENTS AND SCOPE

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

### Example 1: Formulations of Nanoparticle Compositions

### A. Production of Nanoparticle Compositions

In order to investigate safe and efficacious nanoparticle compositions for use in the delivery of mRNA to cells, a range of formulations were prepared and tested. Specifically, the particular elements and ratios thereof in the lipid component of nanoparticle compositions were optimized.

Nanoparticles can be made with mixing processes such as microfluidics and T-junction mixing of two fluid streams, one of which contains the mRNA and the other has the lipid components.

KL22 was prepared according to the procedure in U.S. patent publication no.: 20140162962. Lipid compositions were prepared by combining KL22, a phospholipid (DOPE or DSPC, obtained from Avanti Polar Lipids, Alabaster, Ala.), 1,2-dimyristoyl-sn-glycerol methoxypolyethylene glycol (PEG-DMG, obtained from Avanti Polar Lipids, Alabaster, Ala.), and a structural lipid (cholesterol; Sigma-Aldrich, Taufkirchen, Germany) at concentrations of 50 mM in ethanol. Solutions were stored at −20° C. Lipids were combined to yield desired molar ratios (see Table 2) and diluted with water and ethanol to a final lipid concentration of between 5.5 mM and 25 mM.

Solutions of mRNA at concentrations of 0.1 mg/ml in deionized water were diluted in 50 mM sodium citrate buffer at a pH between 3 and 4 to form a stock solution. The mRNA solution was heated for 2 minutes at 60° C. to denature.

Nanoparticle compositions including mRNA and a lipid component were prepared by combining the lipid solution with the mRNA solution at lipid component to mRNA wt:wt ratios between 5:1 and 50:1. The lipid solution was rapidly injected using a NanoAssemblr microfluidic based system at flow rates between 6 ml/min and 20 ml/min into the mRNA solution to produce a suspension with a water to ethanol ratio between 1:1 and 4:1. The solution was then heated for 2 minutes at 60° C.

Nanoparticle compositions were then processed by dialysis to remove the ethanol and achieve buffer exchange. Formulations were dialyzed twice against phosphate buffered saline (PBS), pH 7.4 at volumes 200 times that of the primary product using Slide-A-Lyzer cassettes (Thermo Fisher Scientific Inc., Rockford, Ill.) with a molecular weight cutoff of 10 kD. The first dialysis was carried out at room temperature for 3 hours. The formulations were then dialyzed overnight at 4° C. The resulting nanoparticle suspension was filtered through 0.2 μm sterile filters (Sarstedt, Nümbrecht, Germany) into glass vials and sealed with crimp closures. Nanoparticle composition solutions of 0.01 to 0.06 mg/ml were generally obtained.

The method described above induces nano-precipitation and particle formation. Alternative processes including, but not limited to, T-junction and direct injection, may be used to achieve the same nano-precipitation.

### B. Characterization of Nanoparticle Compositions

A Zetasizer Nano ZS (Malvern Instruments Ltd, Malvern, Worcestershire, UK) was used to determine the particle size, the polydispersity index (PDI) and the zeta potential of the nanoparticle compositions in 1×PBS in determining particle size and 15 mM PBS in determining zeta potential.

Ultraviolet-visible spectroscopy was used to determine the concentration of mRNA in nanoparticle compositions. 100 μL of the diluted formulation in 1×PBS was added to 900 μL of a 4:1 (v/v) mixture of methanol and chloroform. After mixing, the absorbance spectrum of the solution was recorded between 230 nm and 330 nm on a DU 800 spectrophotometer (Beckman Coulter, Beckman Coulter, Inc., Brea, Calif.). The mRNA concentration in the nanoparticle composition was calculated based on the extinction coefficient of the mRNA used in the composition and on the difference between the absorbance at a wavelength of 260 nm and the baseline value at a wavelength of 330 nm.

A QUANT-IT™ RIBOGREEN® RNA assay (Invitrogen Corporation Carlsbad, Calif.) was used to evaluate the encapsulation of mRNA by the nanoparticle composition. The samples were diluted to a concentration of approximately 5 μg/ml in a TE buffer solution (10 mM Tris-HCl, 1 mM EDTA, pH 7.5). 50 μl of the diluted samples were transferred to a polystyrene 96 well plate and either 50 μl of TE buffer or 50 μl of a 2% Triton X-100 solution was added to the wells. The plate was incubated at a temperature of 37° C. for 15 minutes. The RIBOGREEN® reagent was diluted 1:100 in TE buffer, and 100 μl of this solution was added to each well. The fluorescence intensity was measured using a fluorescence plate reader (Wallac Victor 1420 Multilabel Counter; Perkin Elmer, Waltham, Mass.) at an excitation wavelength of about 480 nm and an emission wavelength of about 520 nm. The fluorescence values of the reagent blank were subtracted from that of each of the samples and the percentage of free mRNA was determined by dividing the fluorescence intensity of the intact sample (without addition of Triton X-100) by the fluorescence value of the disrupted sample (caused by the addition of Triton X-100).

### C. In Vivo Formulation Studies

In order to monitor how effectively various nanoparticle compositions deliver mRNA to targeted cells, different nanoparticle compositions including a particular mRNA (for example, a modified human erythropoietin [hEPO]) are prepared and administered to rodent populations. Mice are intravenously or intramuscularly administered a single dose including a nanoparticle composition of the invention with a formulation such as those provided in Table 2. Dose sizes may range from 0.005 mg/kg to 5 mg/kg, where 5 mg/kg describes a dose including 5 mg of nanoparticle composition for each 1 kg of body mass of the mouse. A control composition including phosphate buffered saline (PBS) may also be employed. Upon administration of nanoparticle compositions to mice, time courses of protein expression, dose responses, and toxicity of particular formulations and doses thereof can be measured by enzyme-linked immunosorbent assays (ELISA). Samples collected from the mice may include blood, sera, and tissue (for example, muscle tissue from the site of an intramuscular injection and internal tissue); sample collection may involve sacrifice of the animals.

Higher levels of protein expression will be indicative of higher mRNA translation and/or nanoparticle composition mRNA delivery efficiencies. As the non-RNA components are not thought to affect translational machineries themselves, a higher level of protein expression is likely indicative of a higher efficiency of delivery of mRNA by a given nanoparticle composition relative to other nanoparticle compositions or the absence thereof.

### D. Process Optimization

Parameters involved in the production of nanoparticle compositions were explored in several dimensions. In one study, a “standard” lipid solution including about 40 mol % KL22, about 20 mol % DOPE, about 38.5 mol % structural lipid, and about 1.5 mol % PEG-DMG was combined with an mRNA under a range of different conditions to examine how production conditions affected the size and encapsulation efficiency of nanoparticle compositions.

The molarity of the lipid solution was varied between 5.5 mM and 25 mM, the rate of injection of the lipid solution into the mRNA solution was varied between 6 ml/min and 20 ml/min, the ratio of water to ethanol in the lipid-mRNA solution was varied between 2:1 and 4:1, and the pH of the citrate buffer was varied between 3 and 4.

The nanoparticle compositions produced in the study were largely insensitive to changes in the preparation process. Regardless of the precise conditions employed, nanoparticle compositions with particle sizes between 95 nm and 105 nm and encapsulation efficiencies greater than 95% were consistently measured. Thus, the process of preparing nanoparticle compositions of the invention is relatively robust.

E. Optimization of Lipid:mRNA Ratios

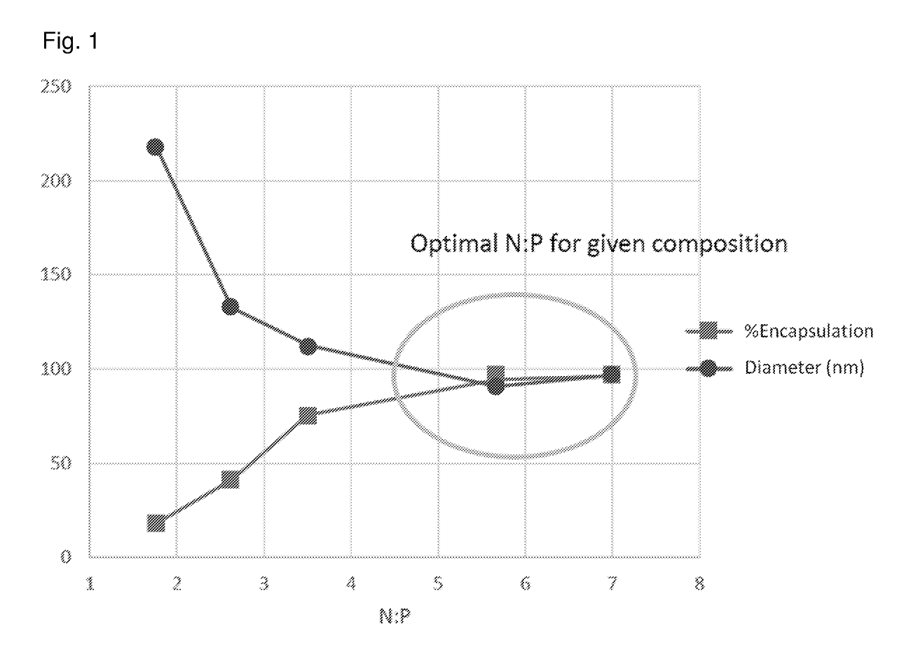

As the N:P ratio of a nanoparticle composition controls both expression and tolerability, nanoparticle compositions with low N:P ratios and strong expression are desirable. N:P ratios vary according to the ratio of lipids to mRNA in a nanoparticle composition. Thus, the wt/wt ratio of total lipid to mRNA was varied between 10:1, 15:1, 20:1, 32:1, and 40:1 for a standard lipid formulation consisting of about 40 mol % KL22, about 20 mol % DOPE, about 38.5 mol % structural lipid, and about 1.5 mol % PEG-DMG. N:P ratios were calculated for each nanoparticle composition assuming a single protonated nitrogen atom. The encapsulation efficiency (EE), size, and polydispersity index of each composition was also measured. The results are summarized in Table 1 and FIG. 1.

TABLE 1
Optimization of lipid:mRNA ratios.

| Lipid:mRNA | | | Size | |
|---|---|---|---|---|
| (wt/wt) | N:P | EE | (nm) | PDI |
| 10:1 | 1.76:1 | 17.9 | 218 | 0.13 |
| 15:1 | 2.63:1 | 41.0 | 133 | 0.068 |
| 20:1 | 3.51:1 | 75.6 | 112 | 0.085 |
| 32:1 | 5.67:1 | 94.3 | 90.7 | 0.15 |
| 40:1 | 7:1 | 96.2 | 96.6 | 0.22 |

Based on these results, it is apparent that higher total lipid:mRNA ratios yield smaller particles with higher encapsulation efficiencies, both of which are desirable. However, the N:P ratio for such formulations exceeds 4. Current standards in the art, for example, nanoparticle compositions including the cationic lipid MC3, DSPC, cholesterol, and PEG-DMG in a 50:10:38.5:1.5 ratio (mol % of lipid component), have N:P ratios of about 5.67. The nanoparticle compositions of the invention may therefore prove efficacious with lower N:P ratios than current standards, and thus be capable of improved expression and tolerability relative to available compositions.

In order to explore the efficacy of nanoparticle compositions with different N:P ratios, the expression of human erythropoietin (hEPO) in mice after low (0.05 mg/kg) or high (0.5 mg/kg) doses of intravenously administered nanoparticle compositions was examined. The concentration of hEPO expressed was measured 6 hours after administration.

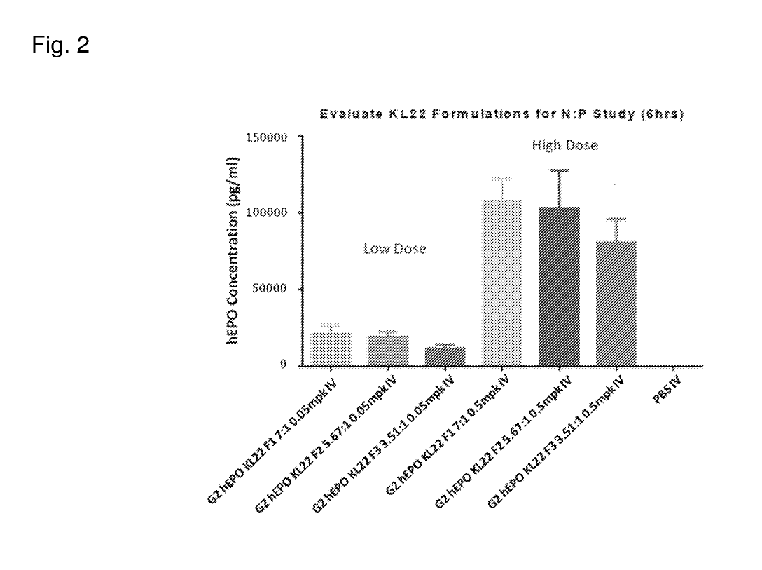

The results of this study are summarized in FIG. 2. Treatment with low doses of nanoparticle compositions yielded low expression (less than 50 ng/ml), while treatment with high doses of nanoparticle compositions produced hEPO concentrations near 100 ng/ml. The expression measured did not vary significantly as the N:P ratio was varied between 3.51:1 and 7:1. Thus, nanoparticle compositions of the invention with lower N:P ratios than those of standards in the art may indeed be efficacious.

### F. Optimization of KL22 Content

As smaller particles with higher encapsulation efficiencies are generally desirable, the relative amounts of various elements in lipid components of nanoparticle compositions were optimized according to these parameters.

In one study, the relative amount of KL22 was varied between 40 mol % and 60 mol % in compositions including DOPE or DSPC as phospholipids to determine the optimal amount of KL22 in the formulations. Formulations were prepared using a standardized process with a water to ethanol ratio in the lipid-mRNA solution of 3:1 and a rate of injection of the lipid solution into the mRNA solution of 12 mL/min on a NanoAssemblr microfluidic based system. This method induces nano-precipitation and particle formation. Alternative processes including, but not limited to, T-junction or direct injection, may also be used to achieve the same nano-precipitation.

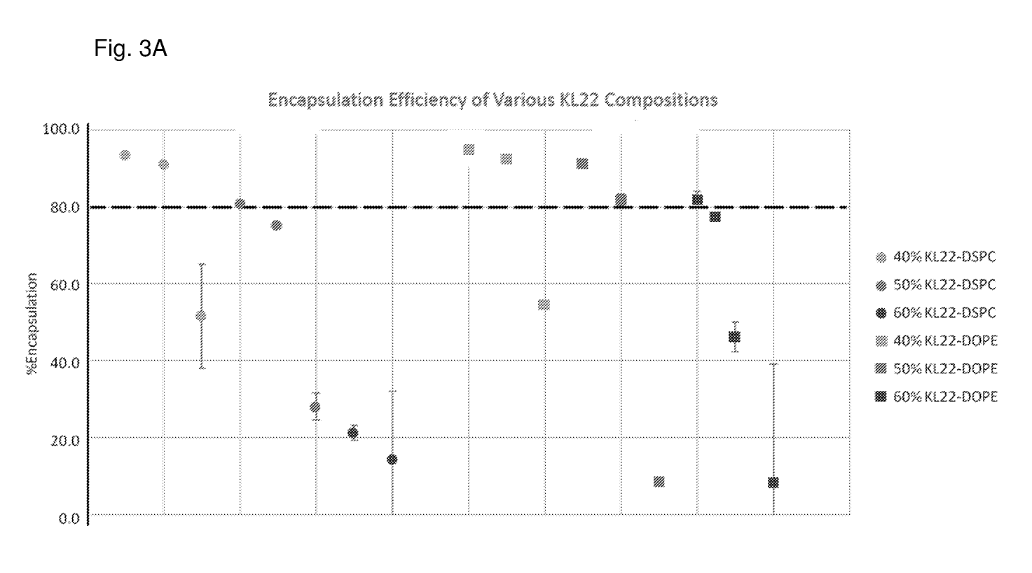

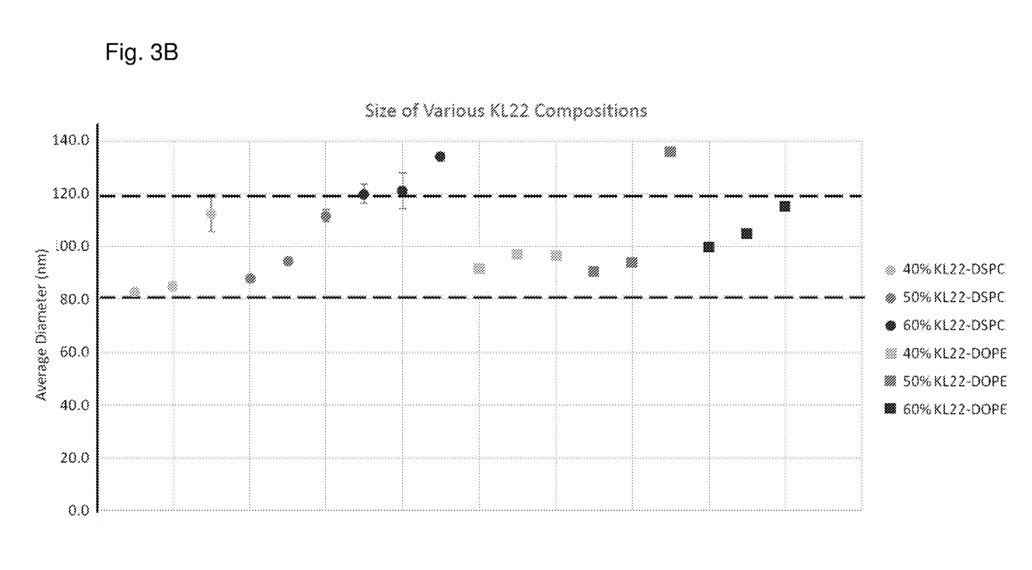

In general, formulations with lower relative amounts of KL22 produced smaller particles with higher encapsulation efficiencies, as shown in FIGS. 3A and 3B. Little difference was seen between those compositions including DOPE compared to those including DSPC, though formulations including DOPE tended to form particles around 100 nm more consistently.

### G. Optimization of Phospholipid Content

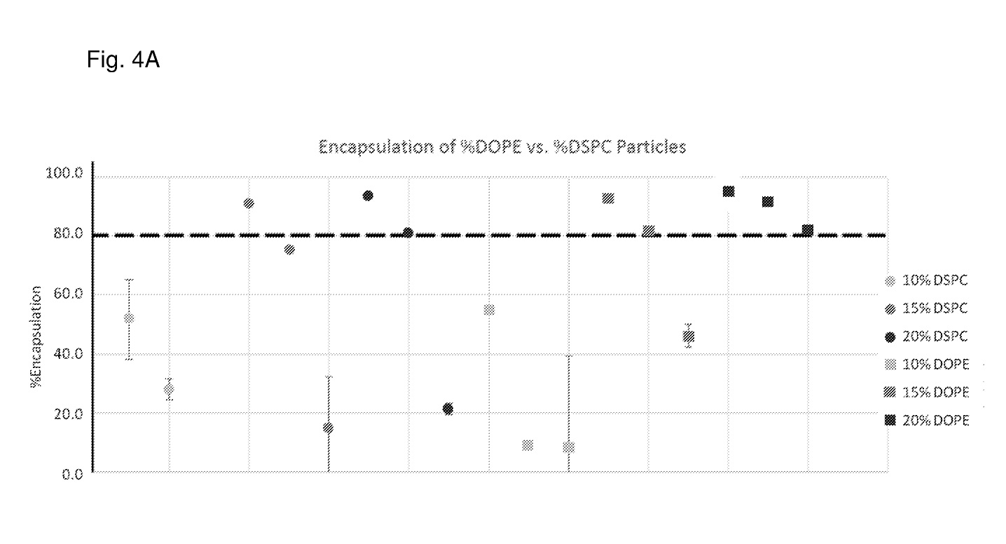

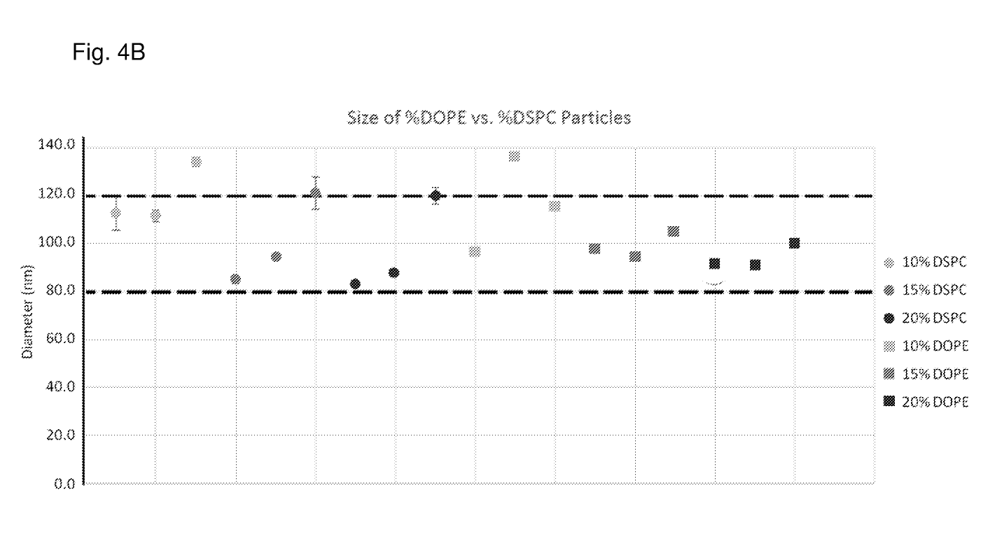

Upon varying the relative amount of phospholipid in a lipid component of a nanoparticle composition between 10 mol % and 20 mol %, it was observed that formulations with higher relative amounts of phospholipid better encapsulated mRNA (FIG. 4A). In particular, those formulations including 20 mol % of DOPE demonstrated superior encapsulation efficiencies. Variation of the content and identify of phospholipid in a nanoparticle composition did not demonstrate a strong effect on particle sizes; however in general it appears that formulations including higher relative amounts of phospholipid produce somewhat smaller particles (FIG. 4B).

### H. Optimization of Cholesterol Content

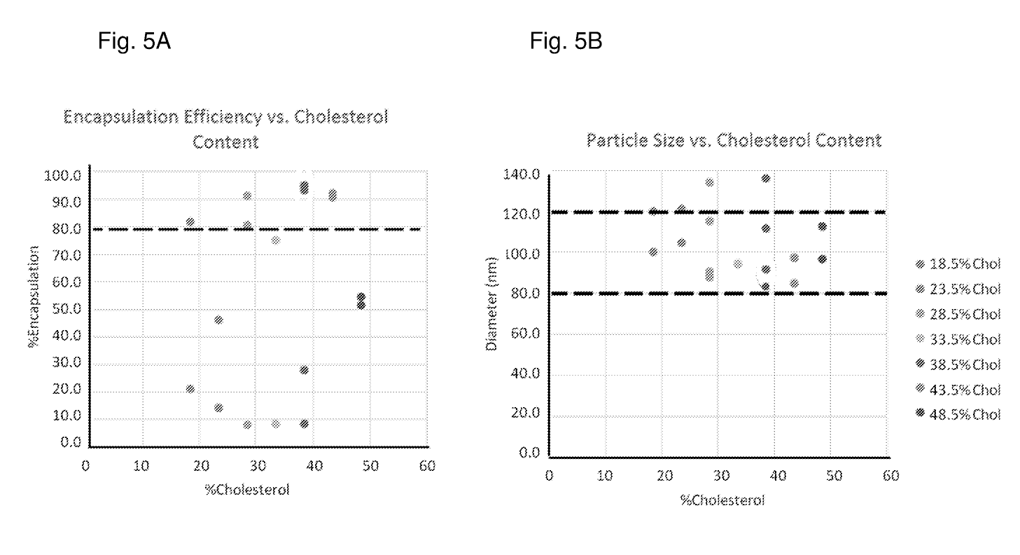

Varying the relative amount of cholesterol in a lipid component of a nanoparticle composition did not produce a notable effect on neither the size nor the encapsulation efficiency of nanoparticle compositions. As shown in FIGS. 5A and 5B, encapsulation efficiencies varied widely with cholesterol content, though particle sizes were clustered between 80 nm and 120 nm regardless of the cholesterol content. However, though the cholesterol content may not demonstrate a strong influence on the physiochemical characteristics of nanoparticle compositions, cholesterol may have important effects in vivo including complement activation and stability effects.

### I. Optimized Formulations

Table 2 summarizes the content and characteristics of several formulations of lipid components useful for nanoparticle compositions of the invention. The formulations included in Table 2 were selected based on having an EE greater than 90% and a particle size between 80 and 100 nm.

TABLE 2
Optimized formulations of lipid components of nanoparticle compositions.

| | Composition | | | Size | |
|---|---|---|---|---|---|
| ID | (mol %) | Components | EE | (nm) | PDI |
| DOPE1 | 40:20:38.5:1.5 | KL22:DOPE:Chol:PEG-DMG | 95.2% | 91.6 | 0.137 |
| DOPE2 | 40:15:43.5:1.5 | KL22:DOPE:Chol:PEG-DMG | 92.3% | 97.3 | 0.165 |
| DSPC1 | 40:20:38.5:1.5 | KL22:DSPC:Chol:PEG-DMG | 93.4% | 82.9 | 0.157 |
| DSPC2 | 40:15:43.5:1.5 | KL22:DSPC:Chol:PEG-DMG | 90.8% | 84.8 | 0.167 |
| DOPE3 | 50:20:28.5:1.5 | KL22:DOPE:Chol:PEG-DMG | 91.3% | 90.4 | 0.118 |

The DOPE1 formulation is the standard referred to in the above examples.

### H. Evaluation of Optimized Formulations

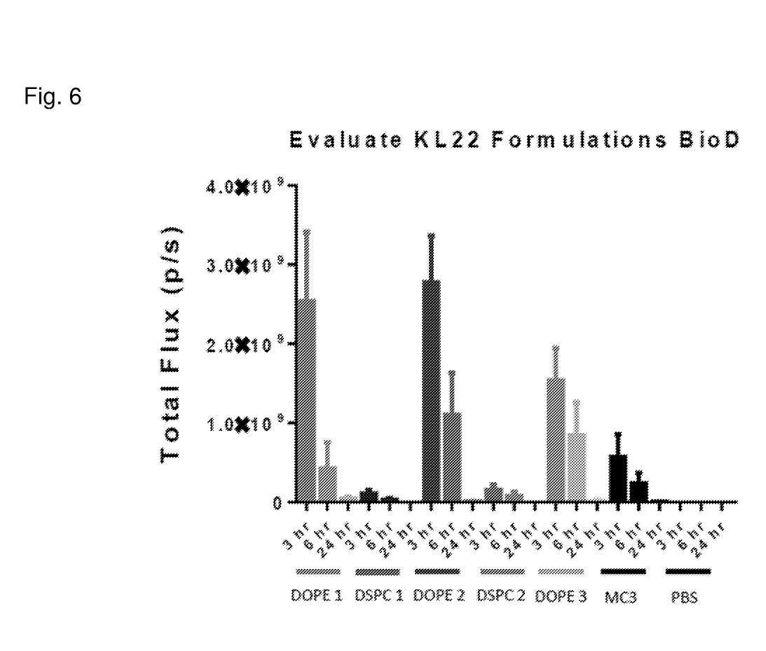

The effectiveness of the KL22 nanoparticle composition formulations presented in Table 2 was evaluated with a bioluminescence study. Formulations including an mRNA encoding modified luciferase were administered to mice and bioluminescence measured at 3, 6, and 24 hour time points. A standard MC3 formulation including about 50 mol % KL22, about 10 mol % DSPC, about 38.5 mol % cholesterol, and about 1.5 mol % PEG-DMG and a PBS control were also tested. As is evident in FIG. 6, the measured total flux was dramatically higher at early time points then at later time points for all formulations. All of the KL22 formulations including DOPE demonstrated significantly higher fluxes than the MC3 formulation, while the KL22 formulations including DSPC exhibited lower fluxes than the MC3 formulation. In particular, the DOPE1 and DOPE2 formulations demonstrated more than double the flux of the MC3 formulation. These results suggest effective and rapid protein expression caused by administration of KL22 formulations relative to administration of other formulations, and indicate that the DOPE1 and DOPE2 formulations may be particularly promising as nanoparticle compositions for use in the delivery of mRNA to cells.

### Example 2: Efficacy of KL22

Nanoparticle formulations including mRNA may include a variety of components including a variety of cationic and/or ionizable lipids. The nanoparticle compositions of the present invention include KL22, however the effectiveness of lipids such as KL10, KL25, and MC3 in nanoparticle compositions has also been explored to varying degrees. Structures of the lipids KL10 and KL25 are described in U.S. patent application publication No. 2014/0162962, while that of MC3 described in U.S. patent application publication No. 2010/0324120.

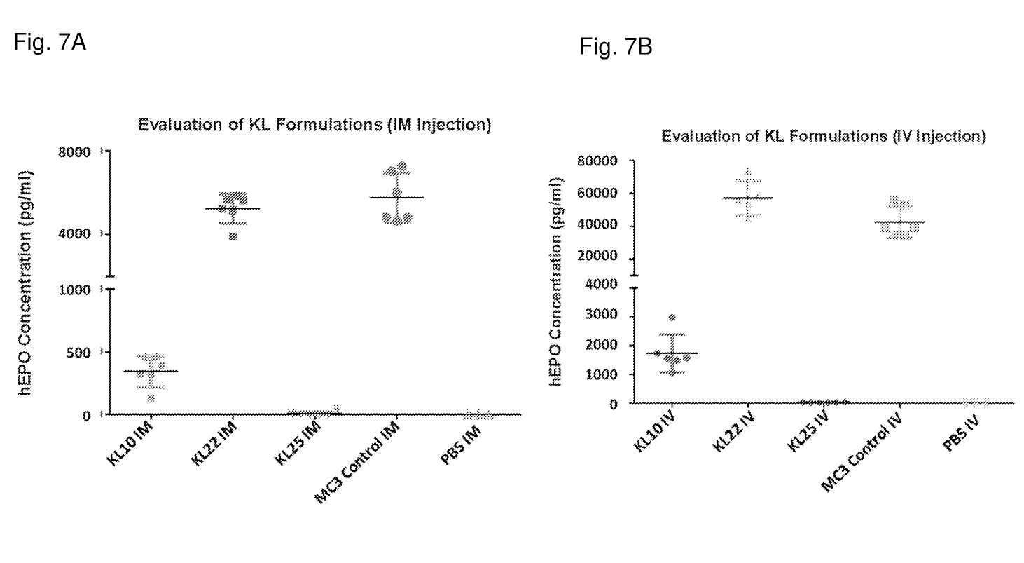

In order to compare the effectiveness of the respective cationic lipids at facilitating mRNA delivery to mammalian cells, mice were intramuscularly or intravenously injected with a single 0.05 mg/kg dose containing a nanoparticle composition including an mRNA encoding modified hEPO and a lipid component including KL10, KL22, KL25, or MC3 and the resultant protein expression measured. A control composition including phosphate buffered saline (PBS) was also employed. FIG. 7A shows results for intramuscular injection, while FIG. 7B displays results for intravenous injection. As is evident in the figures, KL22 and MC3 display similar and far stronger protein expression than other species upon both intravenous and intramuscular injection. Thus, KL22-containing nanoparticle compositions may effectively facilitate the delivery of mRNA to cells.

### Example 3: Mechanism of KL22 Uptake

The method of uptake of KL22-containing nanoparticle compositions by cells was also investigated. Nanoparticle compositions including KL22 or MC3 and an mRNA encoding modified hEPO were administered intramuscularly and intravenously to wild type (WT) mice and mice deficient in low density lipoprotein receptor (LDLR). The DOPE1 formulation was used for KL22 compositions, while the standard MC3 formulation including about 50 mol % KL22, about 10 mol % DSPC, about 38.5 mol % cholesterol, and about 1.5 mol % PEG-DMG. The sizes and encapsulation efficiencies of the two formulations were similar.

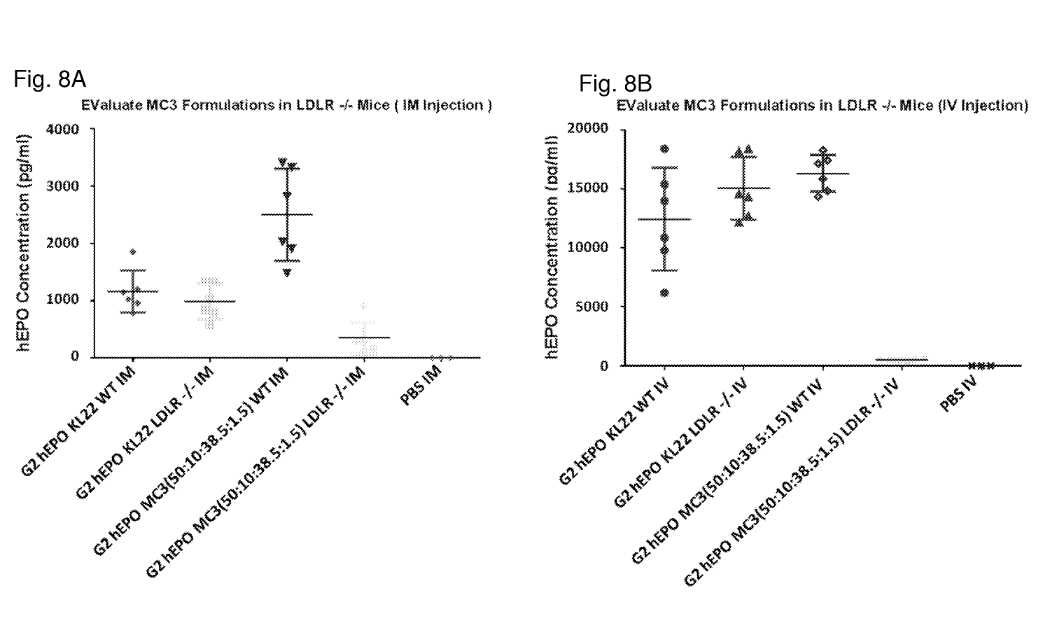

As shown in FIGS. 8A and 8B, the amount of hEPO expressed upon either intramuscular or intravenous administration of KL22-containing nanoparticle compositions was largely independent of the presence or absence of LDLR in the mice. In contrast, protein expression was dramatically lower in mice lacking LDLR for MC3-containing nanoparticle compositions. KL22 therefore operates by a different mechanism than MC3. As LDLRs are often found on the surface of hepatocytes, the LDLR independence observed for KL22-containing nanoparticle compositions may suggest that these compositions do not target hepatocytes via LDL receptors.

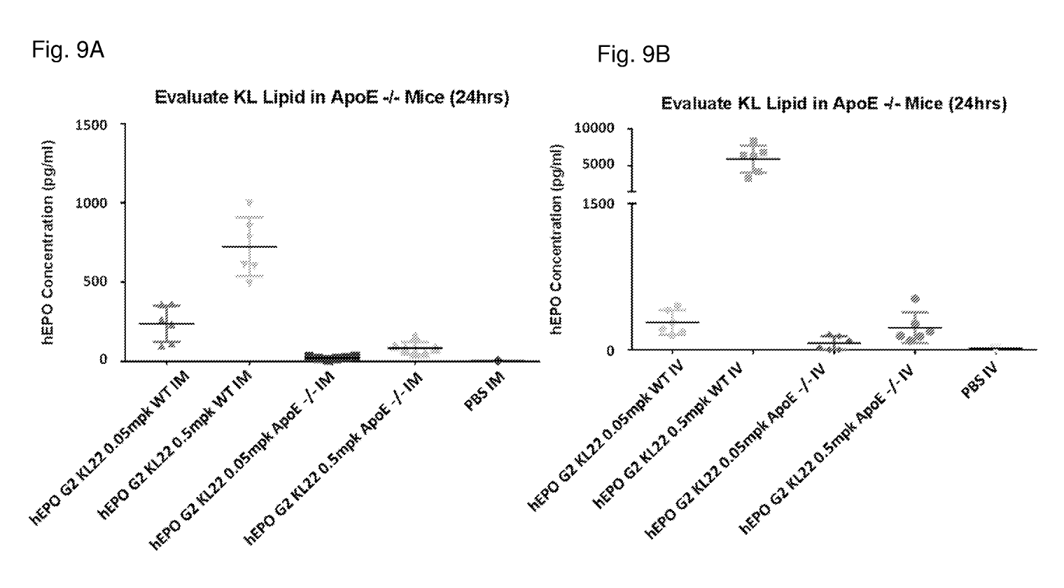

The method of uptake of KL22-containing nanoparticle compositions was further explored by administering compositions to wild type mice and mice deficient in apolipoprotein E (apoE). Doses of 0.05 mg/kg and 0.5 mg/kg were administered both intramuscularly and intravenously. At both dosage levels, protein expression was dramatically lower in apoE deficient mice than in wild type mice (FIGS. 9A and 9B). The effect was particularly pronounced in the high dosage group. The method of uptake for KL22-containing nanoparticle compositions may thus involve interaction with one or more apolipoproteins.

### Example 4: Toxicology and Dose Response

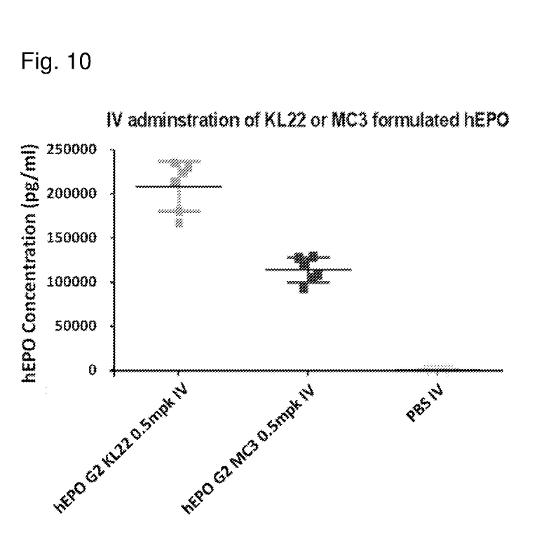

The expression of hEPO and enzyme levels in the liver of mice treated with a single 0.5 mg/kg dose of KL22- or MC3-containing nanoparticle compositions were examined. Administration of a KL22-containing nanoparticle composition resulted in higher hEPO expression after 8 hours than did administration of an MC3-containing nanoparticle composition; both compositions outperformed the PBS control (FIG. 10). Table 3 summarizes the levels of liver enzymes alanine transaminase (ALT) and aspartate transaminase (AST) measured in each instance.

TABLE 3
Comparison of KL22 and MC3 formulations.

| Parameter | KL22 formulation | MC3 formulation |
|---|---|---|
| hEPO expression (ng/ml) | 208 | 112 |
| ALT (ng/ml) | 72.7 | 304 |
| AST (ng/ml) | 108 | 490 |

As is evident in the table, administration of the KL22 formulation provided nearly double the expression of administration of the MC3 formulation. Liver enzymes ALT and AST were also substantially less elevated after administration of the KL22 formulation (4- to 5-fold improvement). The lower elevation of the ALT and AST enzymes seen upon administration of KL22 formulations suggests that KL22-containing nanoparticle compositions have a lower toxicity profile than MC3-containing nanoparticle compositions.

A dose response study was also carried out. Mice were intravenously injected with doses ranging from 0.05 mg/kg to 1.25 mg/kg of formulations including KL22, MC3, or C12-200 (structure available in Love et al., Proc. Natl. Acad. Sci. USA. 2010 107:1864-1869 and Liu and Huang, Molecular Therapy. 2010 669-670). The C12-200 formulation included about 40 mol % KL22, about 30 mol % DOPE, about 25 mol % cholesterol, and about 5 mol % PEG-DMG.

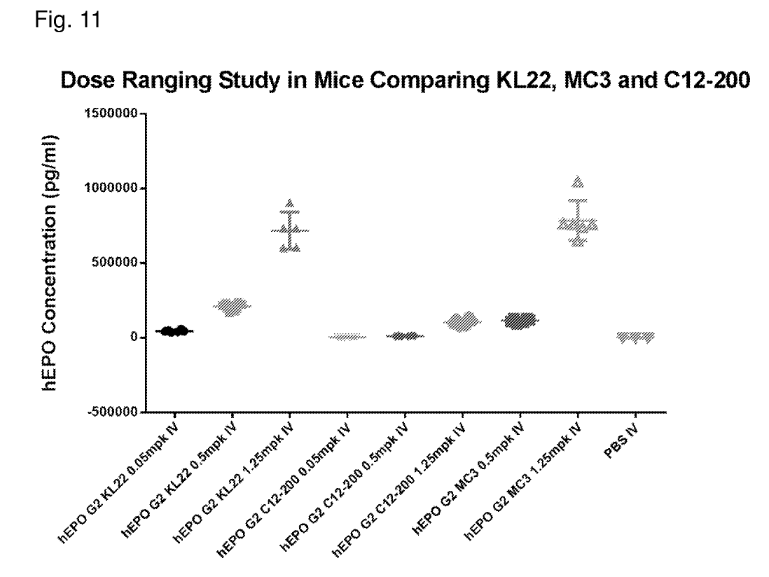

High doses of KL22- and MC3-containing nanoparticle compositions yielded significantly higher hEPO expression than did lower doses and also outperformed the C12-200 (FIG. 11). Though the KL22 and MC3 formulations performed similarly for doses of 1.25 mg/kg, the KL22 formulation performed somewhat better than the MC3 formulation at a dose of 0.5 mg/kg. That KL22 formulations performed substantially better than C12-200 formulations is notable due to the similarities in the structures of the amino lipids.

Table 4 summarizes clinical pathology data measured in the dose response study. As is evident from the results, ALT and AST levels were significantly more elevated for MC3 formulations compared to KL22 formulations, though levels for high KL22 doses were also rather high. This behavior is typical upon administration of high doses of lipid-containing nanoparticles. Creatine phosphokinase (CPK) levels were also significantly more elevated for MC3 formulations. Albumin and total bilirubin levels were low for all formulations, and direct bilirubin was negligible in all cases. The blood urea nitrogen (BUN) to creatinine levels was approximately constant for all formulations.

TABLE 4
Enzyme levels measured after administration of nanoparticle compositions.

| Dose | ALT | AST | CPK | Albumin | Total Bilirubin | BUN | Creatinine |
|---|---|---|---|---|---|---|---|
| KL22 | | | | | | | |
| 0.05 mg/kg | 81.3 | 106.7 | 209.7 | 3.1 | 0.2 | 23.3 | 0.3 |
| 0.5 mg/kg | 72.7 | 108.3 | 136.7 | 3.0 | 0.1 | 24.0 | 0.3 |
| 1.25 mg/kg | 195.0 | 396.0 | 154.7 | 3.1 | 0.2 | 26.3 | 0.3 |
| C12-200 | | | | | | | |
| 0.05 mg/kg | 55.7 | 85.0 | 231.0 | 3.0 | 0.1 | 24.3 | DNR |
| 0.5 mg/kg | 66.7 | 118.7 | 203.7 | 2.8 | 0.2 | 27.0 | 0.3 |
| 1.25 mg/kg | 91.7 | 313.3 | 339.3 | <3 | 0.0 | 27.3 | DNR |
| MC3 | | | | | | | |
| 0.5 mg/kg | 304.0 | 490.3 | 3371.3 | <3 | 0.3 | 29.0 | 0.1 |
| 1.25 mg/kg | 226.3 | 574.0 | 806.7 | <3 | 0.0 | 24.7 | DNR |

The high levels of ALT and AST measured in the high dose group may be suggestive of hepatocyte-specific liver injury, while the elevation of CPK by MC3 formulations may be suggestive of muscular or cardiac injuries. Notably, KL22 formulations provide a safer toxicology profile than MC3 formulations.

### Example 5: Cytokine Induction Profiles

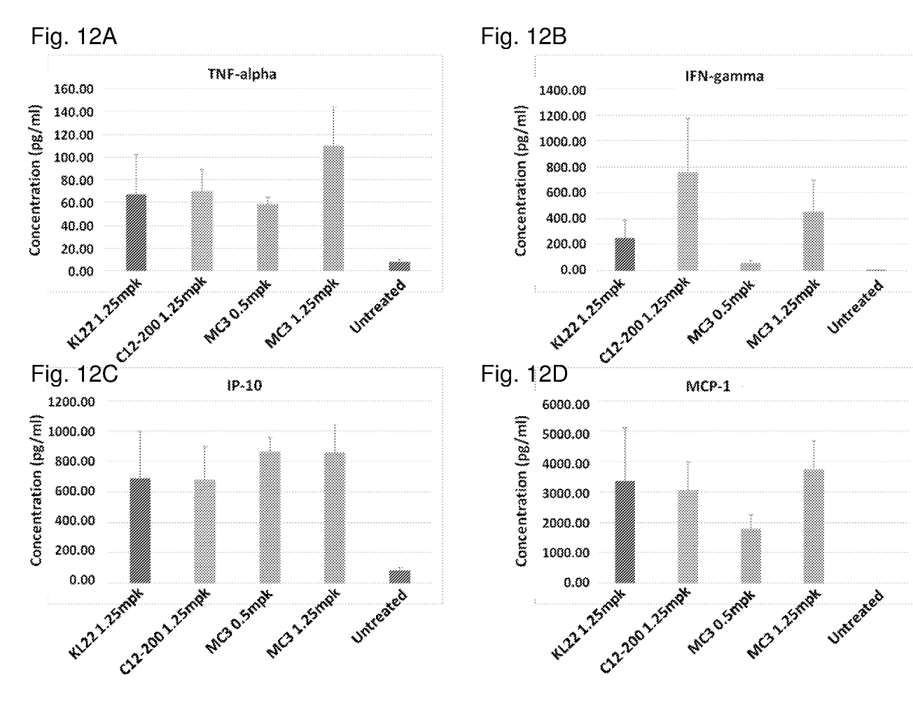

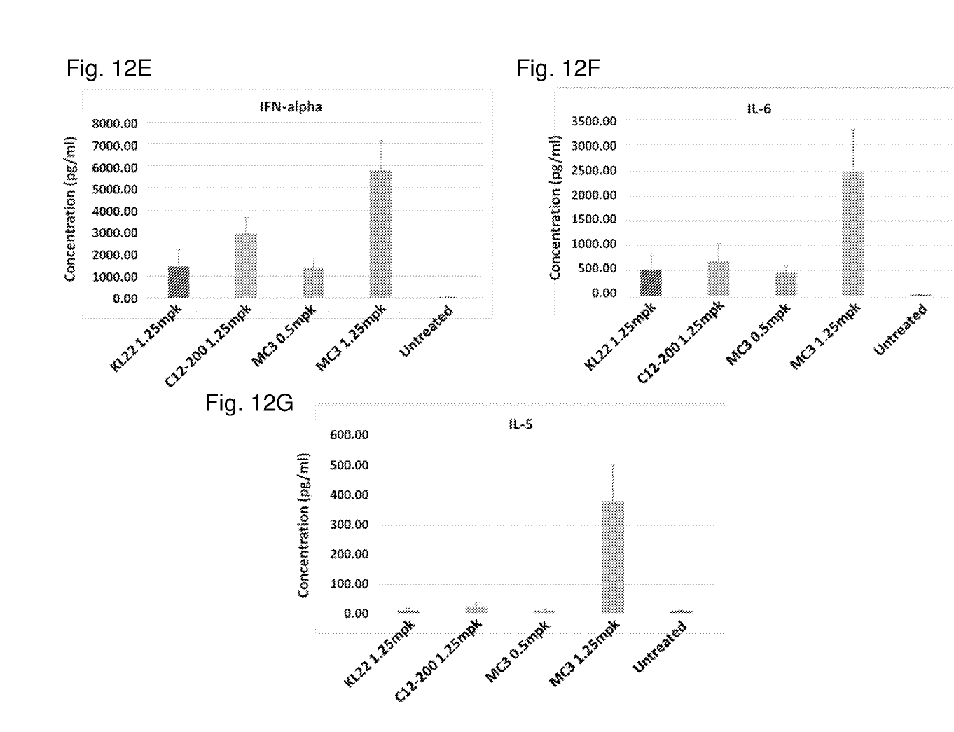

Cytokine expression levels upon administration of nanoparticle compositions including KL22, MC3, and C12-200 were also examined and are summarized in FIGS. 12A-12G. FIGS. 12A, 12B, 12C, 12D, 12E, 12F, and 12G show expression of TNF-α, IFN-γ, IP-10, MCP-1, IFN-α, IL-6, and IL-5, respectively. The data suggests non-specific induction independent of the particular formulation for TNF-α, IFN-γ, IP-10, MCP-1, as well as MCP-3, MIP-1-α, and MIP-1-β. MC3 formulations exclusively induced IL-5, while significantly lower IFN-α and IL-6 induction was observed for KL22 formulations relative to MC3 formulations. Thus, KL22-containing nanoparticle compositions have improved inflammatory profiles relative to nanoparticle compositions including other cationic lipids.

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

1. A method of producing a polypeptide of interest in a mammalian cell, said method comprising contacting said mammalian cell with a nanoparticle composition, said composition comprising (i) a lipid component comprising a compound of formula (I) 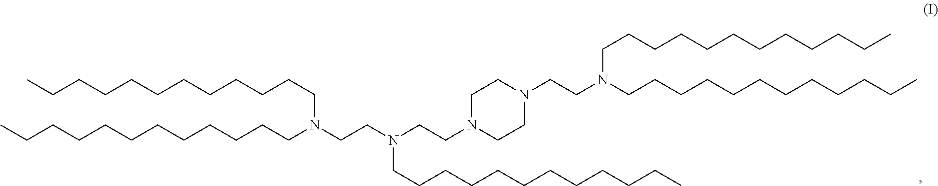 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]
phospholipid, a structural lipid, and a PEG lipid; and (ii) an mRNA encoding said polypeptide of interest, whereby said mRNA is capable of being translated in said cell to produce said polypeptide of interest.

2. A method of delivering an mRNA to a mammalian cell, said method comprising administering to a subject a nanoparticle composition, said composition comprising (i) a lipid component comprising a compound of formula (I), a phospholipid, a structural lipid, and a PEG lipid; and (ii) an mRNA, said administering comprising contacting said mammalian cell with said nanoparticle composition, whereby said mRNA is delivered to said cell.

3. The method of claim 1 or 2, wherein said PEG lipid is selected from the group consisting of a PEG-modified phosphatidylethanolamine, a PEG-modified phosphatidic acid, a PEG-modified ceramide, a PEG-modified dialkylamine, a PEG-modified diacylglyceril, and a PEG-modified dialkylglycerol.

4. The method of any one of claims 1 to 3, wherein said structural lipid is selected from the group consisting of cholesterol, fecosterol, sitosterol, ergosterol, campesterol, stigmasterol, brassicasterol, tomatidine, ursolic acid, and alpha-tocopherol.

5. The method of claim 4, wherein said structural lipid is cholesterol.

6. The method of any one of claims 1 to 5, wherein said phospholipid includes a moiety selected from the group consisting of phosphatidyl choline, phosphatidyl ethanolamine, phosphatidyl glycerol, phosphatidyl serine, phosphatidic acid, 2-lysophosphatidyl choline, and a sphingomyelin.

7. The method of any one of claims 1 to 6, wherein said phospholipid includes one or more fatty acid moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, phytanoic acid, eicosapentaenoic acid, behenic acid, docosapentaenoic acid, and docosahexaenoic acid.

8. The method of any one of claims 1 to 5, wherein said phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

9. The method of claim 8, wherein said phospholipid is DOPE.

10. The method of claim 8, wherein said phospholipid is DSPC.

11. The method of claim 9, wherein said phospholipid is DOPE and said lipid component comprises about 35 mol % to about 45 mol % said compound, about 10 mol % to about 20 mol % DOPE, about 38.5 mol % to about 48.5 mol % structural lipid, and about 1.5 mol % PEG lipid.

12. The method of claim 11, wherein said lipid component comprises about 40 mol % said compound, about 20 mol % DOPE, about 38.5 mol % structural lipid, and about 1.5 mol % PEG lipid.

13. The method of any one of claims 1 to 10, wherein said lipid component comprises about 40 mol % said compound, about 15 mol % phospholipid, about 43.5 mol % structural lipid, and about 1.5 mol % PEG lipid.

14. The method of any one of claims 1 to 10, wherein said lipid component comprises about 45 mol % to about 55 mol % said compound, about 15 mol % to about 25 mol % phospholipid, about 23.5 mol % to about 33.5 mol % structural lipid, and about 1.5 mol % PEG lipid.

15. The method of claim 14, wherein said lipid component comprises about 50 mol % said compound, about 20 mol % phospholipid, about 28.5 mol % structural lipid, and about 1.5 mol % PEG lipid.

16. The method of any one of claims 1 to 15, wherein said mRNA includes one or more of a stem loop, a chain terminating nucleoside, a polyA sequence, a polyadenylation signal, and/or a 5′ cap structure.

17. The method of any one of claims 1 to 16, wherein the wt/wt ratio of said lipid component to said mRNA is from about 5:1 to about 50:1.

18. The method of claim 17, wherein the wt/wt ratio of said lipid component to said mRNA is from about 10:1 to about 40:1.

19. The method of any one of claims 1 to 18, wherein the N:P ratio is from about 2:1 to about 8:1.

20. The method of claim 19, wherein the N:P ratio is from about 2:1 to about 5:1.

21. The method of any one of claims 1 to 20, wherein the mean size of said nanoparticle composition is from about 50 nm to about 150 nm.

22. The method of claim 21, wherein the mean size of said nanoparticle composition is from about 80 nm to about 120 nm.

23. The method of claim 22, wherein the mean size of said nanoparticle composition is about 90 nm.

24. The method of any one of claims 1 to 23, wherein the polydispersity index of said nanoparticle composition is from about 0 to about 0.18.

25. The method of claim 24, wherein the polydispersity index of said nanoparticle composition is from about 0.13 to about 0.17.

26. The method of any one of claims 1 to 25, wherein said nanoparticle composition has a zeta potential of about −10 to about +20 mV.

27. The method of any one of claims 1 to 26, wherein the encapsulation efficiency of said mRNA is at least 50%.

28. The method of claim 27, wherein the encapsulation efficiency of said mRNA is at least 80%.

29. The method of claim 28, wherein the encapsulation efficiency of said mRNA is at least 90%.

30. The method of any one of claims 1 to 29, wherein said nanoparticle composition further comprises a cationic and/or ionizable lipid selected from the group consisting of 3-(didodecylamino)-N1,N1,4-tridodecyl-1-piperazineethanamine (KL10), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,2Z)-octadeca-9,12-dien-1-yloxy]propan-1-amine (Octyl-CLin DMA), (2R)-2-({8-[(3β)-cholest-5-en-3-yloxy]octyl}oxy)-N,N-dimethyl-3-[(9Z,12Z)-octadeca-9,12-dien-1-yl oxy]propan-1-amine (Octyl-CLinDMA (2R)), and (2S)-2-({8-[β)-cholest-5-en-3-yloxy]octyl}oxy)-N,N-dimethyl-3-[(9Z,12Z)-octadeca-9,12-dien-1-yl oxy]propan-1-amine (Octyl-CLinDMA (2S)).

31. The method of any one of claims 1 to 30, wherein said mammalian cell is in a mammal.

32. The method of claim 31, wherein said nanoparticle composition is administered intravenously, intramuscularly, intradermally, subcutaneously, intranasally, or by inhalation.

33. The method of claim 32, wherein a dose of about 0.005 mg/kg to about 5 mg/kg of said nanoparticle composition is administered to said mammal.

34. A nanoparticle composition comprising a lipid component and an mRNA encoding a polypeptide of interest, wherein said lipid component comprises the compound of formula (I), a phospholipid, a structural lipid, and a PEG lipid.

35. The nanoparticle composition of claim 34, wherein said PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, and a PEG-modified diaikylglycerol.

36. The nanoparticle composition of claim 34 or 35, wherein said structural lipid is selected from the group consisting of cholesterol, fecosterol, sitosterol, ergosterol, campesterol, stigmasterol, brassicasterol, tomatidine, ursolic acid, and alpha-tocopherol.

37. The nanoparticle composition of claim 36, wherein said structural lipid is cholesterol.

38. The nanoparticle composition of any one of claims 34 to 37, wherein said phospholipid includes a moiety selected from the group consisting of phosphatidyl choline, phosphatidyl ethanolamine, phosphatidyl glycerol, phosphatidyl serine, phosphatidic acid, 2-lysophosphatidyl choline, and a sphingomyelin.

39. The nanoparticle composition of any one of claims 34 to 38, wherein said phospholipid includes one or more fatty acid moieties selected from the group consisting of lauric acid, myristic acid, myristoleic acid, palmitic acid, palmitoleic acid, stearic acid, oleic acid, linoleic acid, alpha-linolenic acid, erucic acid, arachidic acid, arachidonic acid, eicosapentaenoic acid, behenic acid, docosapentaenoic acid, and docosahexaenoic acid.

40. The nanoparticle composition of any one of claims 34 to 37, wherein said phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanola mine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

41. The nanoparticle composition of claim 40, wherein said phospholipid is DOPE.

42. The nanoparticle composition of claim 40, wherein said phospholipid is DSPC.

43. The nanoparticle composition of claim 41, wherein said phospholipid is DOPE and wherein said lipid component comprises about 35 mol % to about 45 mol % compound of formula (I), about 10 mol % to about 20 mol % DOPE, about 38.5 mol % to about 48.5 mol % structural lipid, and about 1.5 mol % PEG lipid.

44. The nanoparticle composition of claim 43, wherein said lipid component comprises about 40 mol % said compound, about 20 mol % DOPE, about 38.5 mol % structural lipid, and about 1.5 mol % PEG lipid.

45. The nanoparticle composition of any one of claims 34 to 42, wherein said lipid component comprises about 40 mol % compound of formula (I), about 15 mol % phospholipid, about 43.5 mol % structural lipid, and about 1.5 mol % PEG lipid.

46. The nanoparticle composition of any one of claims 34 to 42, wherein said lipid component comprises about 45 mol % to about 55 mol % compound of formula (I), about 15 mol % to about 25 mol % phospholipid, about 23.5 mol % to about 33.5 mol % structural lipid, and about 1.5 mol % PEG lipid.

47. The nanoparticle composition of claim 46, wherein said lipid component comprises about 50 mol % said compound, about 20 mol % phospholipid, about 28.5 mol % structural lipid, and about 1.5 mol % PEG lipid.

48. The nanoparticle composition of any one of claims 34 to 47, wherein said mRNA includes one or more of a stem loop, a chain terminating nucleoside, a polyA sequence, a polyadenylation signal, and/or a 5′ cap structure.

49. The nanoparticle composition of any one of claims 34 to 48, wherein the wt/wt ratio of said lipid component to said mRNA is from about 5:1 to about 50:1.

50. The nanoparticle composition of claim 49, wherein the wt/wt ratio of said lipid component to said mRNA is from about 10:1 to about 40:1.

51. The nanoparticle composition of any one of claims 34 to 50, wherein the N:P ratio is from about 2:1 to about 8:1.

52. The nanoparticle composition of claim 51, wherein the N:P ratio is from about 2:1 to about 5:1.

53. The nanoparticle composition of any one of claims 34 to 52, wherein the mean size of said nanoparticle composition is from about 40 nm to about 150 nm.

54. The nanoparticle composition of claim 53, wherein the mean size of said nanoparticle composition is from about 80 nm to about 120 nm.

55. The nanoparticle composition of claim 54, wherein the mean size of said nanoparticle composition is about 90 nm.

56. The nanoparticle composition of any one of claims 34 to 55, wherein the polydispersity index of said nanoparticle composition is from about 0 to about 0.18.

57. The nanoparticle composition of claim 56, wherein the polydispersity index of said nanoparticle composition is from about 0.13 to about 0.17.

58. The nanoparticle composition of any one of claims 34 to 57, wherein said nanoparticle composition has a zeta potential of about −10 to about +20 mV.

59. The nanoparticle composition of any one of claims 34 to 58, wherein the encapsulation efficiency of said mRNA is at least 50%.

60. The nanoparticle composition of claim 59, wherein the encapsulation efficiency of said mRNA is at least 80%.

61. The nanoparticle composition of claim 60, wherein the encapsulation efficiency of said mRNA is at least 90%.

62. The nanoparticle composition of any one of claims 34 to 61, wherein said nanoparticle composition further comprises a cationic and/or ionizable lipid selected from the group consisting of 3-(didodecylamino)-N1,N1,4-tridodecyl-1-piperazineethanamine (KL10), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3β)-cholest-5-en-3-yloxy]octyl}oxy)-N,N-dimethyl-3-[(9Z,2Z)-octadeca-9,12-dien-1-yloxy]propan-1-amine (Octyl-CLin DMA), (2R)-2-({8-[β)-cholest-5-en-3-yloxy]octyl}oxy)-N,N-dimethyl-3-[(9Z,12Z)-octadeca-9,12-dien-1-yl oxy]propan-1-amine (Octyl-CLinDMA (2R)), and (2S)-2-({8-[(3β)-cholest-5-en-3-yloxy]octyl}oxy)-N, N-di methyl-3-[(9Z,12Z)-octadeca-9,12-dien-1-yl oxy]propan-1-amine (Octyl-CLinDMA (2S)).
